SEQUENCE LISTING (34 sequences)

SEQ ID NO: 1 - protein, 604 aa, Homo sapiens
  SIGNAL 1..24
  REGION 339..381: note = MISC_FEATURE - linear epitope
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 2 - DNA, 1817 bp, Homo sapiens
  source 1..1817: mol_type = genomic DNA
atgatgcgcgcagtgtgggaggcgctggcggcgctggcggcggtggcgtgcctggtgggcgcggtgcgcggcgggcccgggctcagcatgttcgcgggccaggcggcgcagcccgatccctgctcggacgagaacggccacccgcgccgctgcatcccggactttgtcaatgcggccttcggcaaggacgtgcgcgtgtccagcacctgcggccggcccccggcgcgctactgcgtggtgagcgagcgcggcgaggagcggctgcgctcgtgccacctctgcaacgcgtccgaccccaagaaggcgcacccgcccgccttcctcaccgacctcaacaacccgcacaacctgacgtgctggcagtccgagaactacctgcagttcccgcacaacgtcacgctcacactgtccctcggcaagaagttcgaagtgacctacgtgagcctgcagttctgctcgccgcggcccgagtccatggccatctacaagtccatggactacgggcgcacgtgggtgcccttccagttctactccacgcagtgccgcaagatgtacaaccggccgcaccgcgcgcccatcaccaagcagaacgagcaggaggccgtgtgcaccgactcgcacaccgacatgcgcccgctctcgggcggcctcatcgccttcagcacgctggacgggcggccctcggcgcacgacttcgacaactcgcccgtgctgcaggactgggtcacggccacagacatccgcgtggccttcagccgcctgcacacgttcggcgacgagaacgaggacgactcggagctggcgcgcgactcgtacttctacgcggtgtccgacctgcaggtgggcggccggtgcaagtgcaacggccacgcggcccgctgcgtgcgcgaccgcgacgacagcctggtgtgcgactgcaggcacaacacggccggcccggagtgcgaccgctgcaagcccttccactacgaccggccctggcagcgcgccacagcccgcgaagccaacgagtgcgtggcctgtaactgcaacctgcatgcccggcgctgccgcttcaacatggagctctacaagctttcggggcgcaagagcggaggtgtctgcctcaactgtcgccacaacaccgccggccgccactgccattactgcaaggagggctactaccgcgacatgggcaagcccatcacccaccggaaggcctgcaaagcctgtgattgccaccctgtgggtgctgctggcaaaacctgcaaccaaaccaccggccagtgtccctgcaaggacggcgtgacgggtatcacctgcaaccgctgcgccaaaggctaccagcagagccgctctcccatcgccccctgcataaagatccctgtagcgccgccgacgactgcagccagcagcgtggaggagcctgaagactgcgattcctactgcaaggcctccaaggggaagctgaagattaacatgaaaaagtactgcaagaaggactatgccgtccagatccacatcctgaaggcggacaaggcgggggactggtggaagttcacggtgaacatcatctccgtgtataagcagggcacgagccgcatccgccgcggtgaccagagcctgtggatccgctcgcgggacatcgcctgcaagtgtcccaaaatcaagcccctcaagaagtacctgctgctgggcaacgcggaggactctccggaccagagcggcatcgtggccgataaaagcagcctggtgatccagtggcgggacacgtgggcgcggcggctgcgcaagttccagcagcgtgagaagaagggcaagtgcaagaaggcctagcg

SEQ ID NO: 3 - protein, 43 aa, Homo sapiens
VACNCNLHARRCRFNMELYKLSGRKSGGVCLNCRHNTAGRHCH

SEQ ID NO: 4 - DNA, 129 bp, Homo sapiens
  source 1..129: mol_type = genomic DNA
gtggcctgtaactgcaacctgcatgcccggcgctgccgcttcaacatggagctctacaagctttcggggcgcaagagcggaggtgtctgcctcaactgtcgccacaacaccgccggccgccactgccat

SEQ ID NO: 5 - protein, 8 aa, Mus musculus
GYTFTSYN

SEQ ID NO: 6 - protein, 8 aa, Mus musculus
IYPGNGDT

SEQ ID NO: 7 - protein, 9 aa, Mus musculus
ARGGTGFAY

SEQ ID NO: 8 - protein, 6 aa, Mus musculus
QSVSND

SEQ ID NO: 9 - protein, 9 aa, Mus musculus
QQDYSSPWT

SEQ ID NO: 10 - protein, 116 aa, Mus musculus
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARGGTGFAYWGQGTLVTVSA

SEQ ID NO: 11 - protein, 107 aa, Mus musculus
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPWTFGGGTKLEIK

SEQ ID NO: 12 - protein, 440 aa, Mus musculus
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARGGTGFAYWGQGTLVTVSAAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 13 - protein, 214 aa, Mus musculus
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 101 aa, synthetic construct
  REGION 1..101: note = humanized variant
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
EIVMTQSPATLSVSPGERATLSCKASQSVSNDVAWYQQKPGKAPKLLIYYASNRYTGIPPRFSGSGYGTDFTLTINNIESEDAAYYFCQQDYSSPWTFGQG

SEQ ID NO: 15 - protein, 101 aa, synthetic construct
  REGION 1..101: note = humanized variant
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
DIQMTQSPSSLSASVGDRVTITCKASQSVSNDVAWFQQRPGQSPRRLIYYASNRYTGVPSRFSGSGSGTDFTFTISSLEAEDAATYYCQQDYSSPWTFGQG

SEQ ID NO: 16 - protein, 101 aa, synthetic construct
  REGION 1..101: note = humanized variant
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
DIQMTQSPSSLSASVGDRVTITCKASQSVSNDVAWYQQKPGQAPRLLIYYASNRYTGIPPRFSGSGYGTDFTLTINNIESEDAAYYFCQQDYSSPWTFGQG

SEQ ID NO: 17 - protein, 101 aa, synthetic construct
  REGION 1..101: note = humanized variant
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
DIQMTQSPSSLSASVGDRVTITCKASQSVSNDVAWYLQKPGQSPQLLIYYASNRYTGVPSRFSGSGSGTDFTFTISSLEAEDAATYYCQQDYSSPWTFGQG

SEQ ID NO: 18 - protein, 101 aa, synthetic construct
  REGION 1..101: note = humanized variant
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
DIVMTQTPLSLPVTPGEPASISCKASQSVSNDVAWYQQKPGQAPRLLIYYASNRYTGIPPRFSGSGYGTDFTLTINNIESEDAAYYFCQQDYSSPWTFGQG

SEQ ID NO: 19 - protein, 101 aa, synthetic construct
  REGION 1..101: note = humanized variant
  REGION 27..32: note = MISC_FEATURE - CDR according to IGMT
  REGION 50..52: note = MISC_FEATURE - CDR according to IGMT
  REGION 89..97: note = MISC_FEATURE - CDR according to IGMT
EIVMTQSPATLSVSPGERATLSCRASQSVSNDVAWYQQKPGQAPRLLIYYASNRYTGIPARFSGSGSGTEFTLTISSLQSEDFAVYYCQQDYSSPWTFGQG

SEQ ID NO: 20 - protein, 109 aa, synthetic construct
  REGION 1..109: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QVQLVQSGAEVKKPGASVKVSCKASGYTFTSYNMHWVRQATGQGLEWMGAIYPGNGDTSYNQKFKGRVTITADKSTSTAYMELSSLRSEDTAVYYCARGGTGFAYWGQG

SEQ ID NO: 21 - protein, 109 aa, synthetic construct
  REGION 1..109: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QVQLQQSGPGLVKPSQTLSLTCAISGYTFTSYNMHWIRQPPGKGLEWIGAIYPGNGDTSYNQKFKGRVTITADKSTSTAYMELSSLRSEDTAVYYCARGGTGFAYWGQG

SEQ ID NO: 22 - protein, 109 aa, synthetic construct
  REGION 1..109: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QVQLQQSGPGLVKPSQTLSLTCAISGYTFTSYNMHWVRQATGQGLEWMGAIYPGNGDTSYNQKFKGRLTISKDTSKNQVVLTMTNMDPVDTATYYCARGGTGFAYWGQG

SEQ ID NO: 23 - protein, 109 aa, synthetic construct
  REGION 1..109: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
EVQLVQSGAEVKKPGESLRISCKGSGYTFTSYNMHWVRQATGQGLEWMGAIYPGNGDTSYNQKFKGRFTISRDDSKNTAYLQMNSLKTEDTAVYYCARGGTGFAYWGQG

SEQ ID NO: 24 - protein, 109 aa, synthetic construct
  REGION 1..109: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QVQLQESGPGLVKPSQTLSLTCTVSGYTFTSYNMHWVRQAPGQGLEWMGAIYPGNGDTSYNQKFKGRVTISVDTSKNQFSLKLSSVTAADTAVYYCARGGTGFAYWGQG

SEQ ID NO: 25 - protein, 109 aa, synthetic construct; identical to SEQ ID NO: 20
  REGION 1..109: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT

SEQ ID NO: 26 - protein, 109 aa, synthetic construct
  REGION 1..109: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QVQLQQSGPGLVKPSQTLSLTCAISGYTFTSYNMHWVRQATGQGLEWMGAIYPGNGDTSYNQKFKGRVTITADKSTSTAYMELSSLRSEDTAVYYCARGGTGFAYWGQG

SEQ ID NO: 27 - protein, 108 aa, synthetic construct
  REGION 1..108: note = humanized variant
  REGION 26..33: note = MISC_FEATURE - CDR according to IGMT
  REGION 51..58: note = MISC_FEATURE - CDR according to IGMT
  REGION 97..105: note = MISC_FEATURE - CDR according to IGMT
QVQLVQSGAEVKKPGASVKVSCKASGYTFTSYNMHWVRQAPGQGLEWMGAIYPGNGDTSYAQKFKGRVTMTRDTSTSTVYMELSSLRSEDTAVYYCARGGTGFAYWGQ

SEQ ID NO: 28 - protein, 5 aa, Mus musculus
SYNMH

SEQ ID NO: 29 - protein, 17 aa, Mus musculus
AIYPGNGDTSYNQKFKG

SEQ ID NO: 30 - protein, 7 aa, Mus musculus
GGTGFAY

SEQ ID NO: 31 - protein, 11 aa, Mus musculus
KASQSVSNDVA

SEQ ID NO: 32 - protein, 7 aa, Mus musculus
YASNRYT

SEQ ID NO: 33 - protein, 22 aa, Homo sapiens
ARRCRFNMELYKLSGRKSGGVC

SEQ ID NO: 34 - DNA, 66 bp, Homo sapiens
  source 1..66: mol_type = genomic DNA
gcccggcgctgccgcttcaacatggagctctacaagctttcggggcgcaagagcggaggtgtctgc

(19) United States
(12) Patent Application Publication
(10) Pub. No.: US 2025/0215071 A1
(43) Pub. Date: Jul. 3, 2025

(54) Novel Mcl-1 inhibitor and combination of Mcl-1 and a BH3 mimetic, such as a Bcl-2 inhibitor

(71) Applicant: NETRIS PHARMA, LYON (FR)

(72) Inventors: Andrea PARADISI, SAINT QUENTIN FALLAVIER (FR); Patrick MEHLEN, SEREZIN-DE-LA-TOUR (FR); Ambroise MANCEAU, MOULAY (FR); Lisa FRYDMAN, NOISIEL (FR); David NEVES, VERNAISON (FR)

(21) Appl. No.: 18/850,214

(22) PCT Filed: Mar. 29, 2023
(86) PCT No.: PCT/EP2023/058113

§ 371 Date: Sep. 24, 2024

(30) Foreign Application Priority Data
Mar. 29, 2022 (EP) 22305395.0

(51) Int. Cl. C07K 16/22; A61K 31/496; A61K 31/5377; A61K 39/00; A61P 35/00; C07K 16/28

(52) U.S. Cl. CPC C07K 16/22; A61K 31/496; A61K 31/5377; A61P 35/00; C07K 16/2803; A61K 2039/505; C07K 2317/31; C07K 2317/34; C07K 2317/51; C07K 2317/515; C07K 2317/565; C07K 2317/76

(57) ABSTRACT

In netrin-1 expressing tumor cells, netrin-1 interference triggers Mcl-1 degradation, restricted to these netrin-1 expressing tumor cells. It is proposed a netrin-1 interference to trigger tumor-specific Mcl-1 degradation. In addition, netrin-1 interference may be combined with BH3-mimetics, especially a Bcl-2 inhibitor. Netrin-1 interference is made using anti-netrin-1 antibodies. The invention proposes an anti-netrin-1 antibody for use as a tumor-specific Mcl-1 inhibitor in the treatment of a Mcl-1+ tumor, more particularly a Mcl-1+, netrin-1+ and netrin-1 receptor+, especially UNC5B+, tumor. Also provided is a combination of an anti-netrin-1 antibody and a Bcl-2 inhibitor, such as Venetoclax.

The present invention relates to novel Mcl-1 inhibitors and to a combination of such a Mcl-1 inhibitor and a BH3 mimetic, such as a Bcl-2 inhibitor. The invention also concerns pharmaceutical compositions, combined compositions or agent combinations for use in treating cancer, and methods to treat cancer.

### BACKGROUND

The balance between cellular survival and death is crucial for the homeostasis of organisms. This balance is controlled at least in part by the interactions of the Bcl-2 family proteins, consisting of pro-apoptotic effectors Bax, Bak and Bok, pro-apoptotic BH3-only proteins (including those members: Bad, Bid, Puma, Bim, and Noxa), and anti-apoptotic proteins of the Bcl-2 family (including Bcl-2, Bcl-XL, Mcl1, Bcl-W, Bfl-1/A1) (Singh et al., 2019). In cancer, the equilibrium of this balance is shifted towards pro-survival Bcl-2 members, that are often overexpressed and stabilized (Delbridge et al., 2016). Hence, Bcl-2 members allow cancer cells to evade cell death, which is a crucial step in the acquisition of the malignant phenotype (Hanahan and Weinberg, 2000; Juin et al., 2013). Cells protected by anti-apoptotic Bcl-2 members are often “primed” for apoptosis, because of oncogenic stress and stabilization of BH3-only proteins (Adams and Cory, 2018; Mérino et al., 2012). Thus, therapeutic strategies targeting anti-apoptotic Bcl-2 proteins are relevant, and selective and potent inhibitors of their members have been developed over the past few decades (Merino et al., 2018). Such inhibitors, named “BH3 mimetics”, are mainly effective in the treatment of hematopoietic cancers which display high levels of Bcl-2 family proteins (Ewald et al., 2019; Scheffold et al., 2018). However, it is possible that a combination of different BH3 mimetics may be effective to treat a broader panel of tumors, including solid tumors, which rely on more than one Bcl-2 protein for their survival (Soderquist et al., 2018). To date, only the Bcl-2-specific inhibitor Venetoclax/ABT199 has been clinically approved (Souers et al., 2013).

A few molecules targeting the other members of the Bcl-2 family have reached the clinic, such as the Mcl-1 inhibitor S63845 (Kotschy et al., 2016). BH3-mimetics targeting Mcl-1 have thus been developed (S63845, AMG 176, and AMG 397). However, the clinical development of these compounds, in particular those targeting Mcl-1, has been delayed because of on-target effects on healthy cells (Merino et al., 2018). Indeed, the presence of this anti-apoptotic protein is crucial in many tissues and cell types, including hematopoietic stem cells, thymus epithelium, mature and immature B and T lymphoid cells, activated T cells, cardiomyocytes, neural cells and hepatocytes (Merino et al., 2018). Preliminary tests on humanized Mcl-1 mice with S63845 revealed a decrease in the B-cell population during treatment, but an overall good tolerability (Brennan et al., 2018). Nonetheless, the recent suspension of a clinical study of two Mcl-1 inhibitors, AMG 176 (NCT02675452) and AMG 397 (NCT03465540), evaluating possible cardiac toxicity of these compounds, still raises some safety concerns (Wei et al., 2020). There are several reports regarding biochemical limitations and relative toxicity of these Mcl-1 inhibitors on healthy hematopoietic cells, that have hindered the development of these therapeutics (Ashkenazi et al., 2017; Opferman et al., 2005; Zhang et al., 2007). Therefore, new leads that allow the targeting of specific members of the Bcl-2 family directly in the tumors are of great interest.

UNC5B/UNC5H2 (UNCoordinated Homolog 5), a type I transmembrane receptor discovered by its effect on neuronal navigation during embryonic development, is a receptor able to actively induce apoptosis when unbound to its ligand, netrin-1 (Llambi et al., 2001). This characteristic defines all members of the dependence receptor family, which activate dual signaling pathways: in the presence of their respective ligands, they induce a “positive” signaling pathway, such as signals of proliferation or migration, whereas in the absence of their specific ligands, they activate a “negative” signaling pathway leading to programmed cell death (Mehlen and Bredesen, 2004, 2011; Negulescu and Mehlen, 2018). Hence, the expression of these receptors renders the cell dependent on the presence of their respective ligands for its survival (Bredesen et al., 2005; Mehlen and Puisieux, 2006). The dependence receptor family consists of more than 20 members (Goldschneider and Mehlen, 2010), including netrin-1 receptors UNC5H (i.e., UNC5H1/A, UNC5H2/B, UNC5H3/C and UNC5H4/D) and DCC (Llambi et al., 2001; Mehlen et al., 1998).

UNC5B, along with netrin-1, exerts pleiotropic effects, ranging from axon guidance and migration to angiogenesis (Castets et al., 2009; Lai Wing Sun et al., 2011). However, owing to its death domain, UNC5B has been extensively described as a pro-death receptor, a death activity blocked through multimerization upon binding of netrin-1. As such, UNC5B has been defined as a conditional tumor suppressor, and netrin-1 as a pro-oncogenic factor (Llambi et al., 2001; Thiebault et al., 2003). Compatible with this concept, netrin-1, which is a protein expressed mainly during embryonic development, has been shown to be re-expressed by cancer cells as well as by the tumor microenvironment in a large fraction of human neoplasms (Mehlen et al., 2011; Sung et al., 2019). This has been shown to occur in inflammatory-associated colorectal cancer (Paradisi et al., 2008; Paradisi et al., 2009), metastatic breast cancer (Fitamant et al., 2008), lung cancer (Delloye-Bourgeois et al., 2009a), neuroblastoma (Delloye-Bourgeois et al., 2009b), lymphoma (Broutier et al., 2016), and melanoma (Boussouar et al., 2020). In these models, interference between netrin-1 and its receptors was sufficient to trigger cancer cell death and to induce tumor growth inhibition. Based on these findings, a monoclonal antibody neutralizing netrin-1 and blocking the netrin-1/UNC5B interaction, dubbed NP137, was developed (Grandin et al., 2016) and is currently being assessed in several phase I and II clinical trials (ClinicalTrials.gov identifiers NCT02977195 and NCT04652076).

There is thus the need for novel Mcl-1 inhibitors that are both efficacious and present a satisfactory safety profile. There is also the need for novel combination cancer therapies based on combination partners, which show a synergistic effect providing the advantage of substantially increased long-term efficacy and improved safety profile. It is further interesting for the combination to allow reducing the efficacy dose of one or the two combined agents, especially the BH3 mimetic or the Bcl-2 inhibitor, especially for safety reasons. It would further be of uppermost interest to have agents, especially the Mcl-1 inhibitor, that are tumor specific, in order to limit secondary effects on healthy tissues.

### SUMMARY OF INVENTION

Little is known about the mechanism by which apoptotic signaling is triggered by UNC5B. Upon netrin-1 deprivation, UNC5B adopts an open conformation, allowing phosphatase PP2A to catalyze dephosphorylation of DAPK (serine/threonine Death-Associated Protein Kinase), which in turn induces cell death by still unknown mechanisms (Guenebeaud et al., 2010; Llambi et al., 2005; Wang et al., 2009). In the present work, the CRL3-COMMD2 complex has been identified as a crucial mediator of UNC5B-induced apoptosis. It was completely unexpected that in the presence of disengaged UNC5B (not bound to netrin-1), this CRL3-COMMD2 complex ubiquitinates the anti-apoptotic Bcl-2-member Mcl-1, targeting it for proteasomal degradation. Consequently, decreased levels of Mcl-1 triggered mitochondrial depolarization and activation of the intrinsic apoptotic pathway. “Netrin-1 interference” comprises impeding netrin-1 binding to UNC5B. This can be effected by providing an anti-netrin-1 specific antibody. Prior knowledge was that dependence receptors such as the netrin-1 receptors triggers cell death independently of the mitochondrial dependent pathway. It was thus completely unexpected that, in netrin-1 expressing tumor cells, netrin-1 interference could trigger Mcl-1 degradation. It was further completely unexpected to find a means capable of inducing a Mcl-1 degradation that is restricted to these netrin-1 expressing tumor cells, and thus to be able to propose netrin-1 interference to trigger tumor-specific Mcl-1 degradation in Mcl-1+ tumors. The present work provides preclinical evidence that NP137 may be used as a specific Mcl-1 inhibitor in tumor patients, and be used in combination with BH3-mimetics. This allow overcoming toxicity that is otherwise associated with this type of compound. Data presented herein that other netrin-1 receptors, in particular UNC5A and UNC5C, are present in Mcl1+ tumor cells and could be involved in the present invention as well.

An object of the invention is thus an anti-netrin-1 antibody, especially an anti-netrin-1 monoclonal antibody, for use as a tumor-specific Mcl-1 inhibitor in the treatment of a Mcl-1+ tumor. Another object of the invention is the use of an anti-netrin-1 antibody, especially an anti-netrin-1 monoclonal antibody, for the manufacture of a medicament for use as a tumor-specific Mcl-1 inhibitor in the treatment of a Mcl-1+ tumor.

Another object of the invention is a method for inhibiting Mcl-1 specifically in Mcl-1+ tumor, in patient having such tumor, comprising administering said patient with an efficient amount of an anti-netrin-1 antibody, especially an anti-netrin-1 monoclonal antibody, exerting a tumor-specific Mcl-1 inhibition. In an object, the invention is a method of treatment of a patient suffering from a Mcl-1+ tumor, comprising administering said patient with an efficient amount of an anti-netrin-1 antibody, especially an anti-netrin-1 monoclonal antibody, and exerting a tumor-specific Mcl-1 inhibition or degradation.

As disclosed herein, the Mcl-1+ tumor or cancer may also be one expressing UNC5B (UNC5B+ tumor or cancer) and netrin-1 (netrin-1+ tumor or cancer).

As disclosed herein, the Mcl-1+ tumor or cancer may also be one expressing UNC5B (UNC5B+ tumor or cancer) and/or another netrin-1 receptor, such as UNC5A and/or UNC5C. Said tumor or cancer may also express netrin-1 (netrin-1+ tumor or cancer).

“Tumor-specific” in the present document means that the antibody induces Mcl-1 inhibition or degradation specifically in tumor cells (“restricted to Mcl-1+ tumor cells”). The antibody does not induce this inhibition in healthy cells containing or expressing Mcl-1 (“healthy Mcl-1+ cells”). Indeed, it has been documented above that, for example, the presence of this anti-apoptotic protein Mcl-1 is crucial in many tissues and cell types, including hematopoietic stem cells, thymus epithelium, mature and immature B and T lymphoid cells, activated T cells, cardiomyocytes, neural cells and hepatocytes. Thus, the antibody does not induce this inhibition in these cells.

In an aspect, the anti-netrin-1 antibody, especially the anti-netrin-1 monoclonal antibody, is for use as a tumor-specific Mcl-1 inhibitor in tumor cells expressing Mcl-1, netrine-1 and UNC5B.

In an aspect, the consequence of the anti-netrin-1 antibody, especially an anti-netrin-1 monoclonal antibody, binding to netrin-1, is that UNC5B is disengaged from netrin-1 or cannot be engaged with netrin-1 and this results in Mcl-1 inhibition or degradation. In another aspect, disengaged or unbound UNC5B makes that the CRL3-COMMD2 complex ubiquitinates the anti-apoptotic Bcl-2-member Mcl-1, targeting it for proteasomal degradation. In another aspect, the consequence of the anti-netrin-1 antibody, especially an anti-netrin-1 monoclonal antibody, binding to netrin-1, is that UNC5B provokes Mcl-1 degradation.

In still another aspect, all this occurs in tumors expressing netrin-1 i.e. the tumor cells and/or the tumor environment express netrin-1. In still another aspect, all this occurs in tumors expressing netrin-1 and a netrin-1 receptor, especially UNC5B.

Thus, the invention proposes an anti-netrin-1 antibody, especially an anti-netrin-1 monoclonal antibody, for use as a tumor-specific Mcl-1 inhibitor in the treatment of a Mcl-1+ tumor, more particularly a tumor expressing Mcl-1, netrin-1 and a netrin-1 receptor, especially a Mcl-1+, netrin-1+ and UNC5B+ tumor, as disclosed herein.

In an aspect, the antibodies are used, or for use, to trigger tumor-specific Mcl1 degradation and induce tumor-specific cell death.

Another object of the invention is a combination of an anti-netrin-1 antibody, or an antigen-binding fragment thereof, and an inhibitor of an anti-apoptotic protein of the Bcl-2 family, preferably a Bcl-2 inhibitor, for use in the treatment of a Mcl1+ cancer or of a Mcl1+ and UNC5B+ cancer, or a Mcl1+ and netrin-1 receptor+ cancer (especially a receptor selected from UNC5A, B and C). As disclosed herein, the Mcl-1+ tumor or cancer may also 15 be one expressing UNC5B (UNC5B+ tumor or cancer) and netrin-1 (netrin-1+ tumor or cancer).

Another object of the invention is a combination, a pharmaceutical combination, or an anti-cancerous combination, comprising
(a) an anti-netrin-1 antibody, especially an anti-netrin-1 monoclonal antibody, and
(b) an inhibitor of an anti-apoptotic protein of the Bcl-2 family, preferably a Bcl-2 inhibitor,
for simultaneous, sequential or separate use.

Herein, (b) relates to a BH3 mimetic, as an inhibitor of anti-apoptotic protein(s) of the Bcl-2 family. This includes inhibitors of the Bcl-2 protein, which is one member of the anti-apoptotic proteins Bcl-2 family.

In an aspect, this combination is for use in the treatment of a cancer, with (a) and (b) intended for simultaneous, sequential or separate administration to a patient.

Another object of the invention is a pharmaceutical composition, or an anti-cancerous composition, comprising
(a) an anti-netrin-1 antibody, especially an anti-netrin-1 monoclonal antibody,
(b) an inhibitor of an anti-apoptotic protein of the Bcl-2 family, preferably a Bcl-2 inhibitor,
and
(c) a pharmaceutically acceptable vehicle or excipient.

Another object of the invention is a pharmaceutical composition kit, comprising
(a) a first vial or container comprising an anti-netrin-1 antibody, especially an anti-netrin-1 monoclonal antibody,
(c) a second vial or container comprising an inhibitor of an anti-apoptotic protein of the Bcl-2 family, preferably a Bcl-2 inhibitor, and
(b) possibly a notice with instructions concerning the administration regimen, such as instructions to proceed with a simultaneous, sequential or separate administration to a patient of all or part of the content of both vials or containers.

Another object of the invention is the use of (a) an anti-netrin-1 antibody, especially an anti-netrin-1 monoclonal antibody, and (b) an inhibitor of an anti-apoptotic protein of the Bcl-2 family, preferably a Bcl-2 inhibitor, for the manufacture of a medicament or pharmaceutical combination for use in the treatment of a cancer. In an aspect, (a) and (b) are intended for simultaneous, sequential or separate administration to a patient.

Another object of the invention is a method for treating a cancer in a patient in need thereof, comprising administering said patient with an efficient amount of (a) an anti-netrin-1 antibody, especially an anti-netrin-1 monoclonal antibody, and (b) an inhibitor of an anti-apoptotic protein of the Bcl-2 family, preferably a Bcl-2 inhibitor. This administration may be made under the form of different administration regimens. It can comprise a simultaneous, a sequential, or a separate administration of (a) and (b) to said patient. The patient in need thereof is a patient having a tumor as disclosed herein.

In these different objects relative to the combined use of (a) and (b), the anti-netrin-1 antibody is a tumor-specific Mcl-1 inhibitor. This antibody provides for the function of Mcl-1 inhibition or degradation, in a tumor specific way, as explained herein. Thus, this function is now combined to the anti-apoptotic Bcl-2 protein(s) inhibition known to the skilled person. However, at the difference with the prior anti-Mcl-1/anti-Bcl-2 combinations, the present one beneficiates from a Mcl-1 inhibition that is restricted to the tumor cells as disclosed herein. In other words, the Bcl-2 inhibitor or the BH3 mimetic in the combination may be any efficient Bcl-2 inhibitor (at the exclusion of a Mcl-1 inhibitor), and it can be currently clinically approved Bcl-2 inhibitors or BH3 mimetics or future clinically approved Bcl-2 inhibitors or BH3 mimetics. Inhibitors of the Bcl-2 anti-apoptotic protein family are usually called BH3 mimetics, and can comprise Bcl-2, Bcl-XL, Bcl-W, Bfl-1/A1 proteins. It can be an inhibitor of Bcl-2 itself (Bcl-2 inhibitor) or an inhibitor of any other members of the Bcl-2 family. It can also be an inhibitor of at least two of these proteins, for example inhibitor of Bcl-2 and Bcl-XL, or of Bcl-2, Bcl-W and Bfl-1/A1.

The present combination of two BH3 mimetics or Bcl-2 anti-apoptotic proteins family inhibitors is deemed preventing Bcl-2-family member compensation and enhancing cancer cell death. It is here demonstrated, that combined treatment with anti-netrin-1 neutralizing antibody acting as a tumor-specific Mcl-1 inhibitor and the Bcl-2 inhibitor Venetoclax strongly reduced tumor growth in murine models and triggered apoptosis in human breast cancer ex vivo slices. Combination of netrin-1 silencing and Navitoclax quickly and strongly triggered apoptosis as well. This is the first time that such a combination is proposed, incorporating one tumor-specific inhibitor and the present data supports that combining netrin-1 interference with BH3 mimetics triggers cancer cell death efficiently, and with reduced side effects, and thus offers potential therapeutic benefit.

Venetoclax is a clinically accepted BH3 mimetic. In an embodiment, Venetoclax is used in the combination of the invention. In another embodiment, Navitoclax is used as BH3 mimetic in the combination of the invention.

In an aspect, the Mcl-1+ tumor or cancer is especially one having a percentage of Mcl-1 positive tumor cells which is equal to or above grade 3, or equal to or above grade 4, or equal to grade 5 (see hereinafter the method of determination of the Mcl1+ tumors).

### DETAILED DESCRIPTION

Cancers Concerned with the Invention

R. S. Soderquist et al. 2018 show that there are various dependencies or co-dependencies of diverse cancers on BCL-2 genes, and that most cell lines depend on at least one combination of Bcl-2, Bcl-XL, and Mcl-1 inhibitors for survival. The authors develop the idea of a roadmap for rationally targeting Bcl-2 family dependencies in diverse human cancers.

The present invention primarily concern a particular group of cancers, which characterize by Mcl-1 acting as a pro-survival Bcl-2 member and, unexpectedly, in relation with the presence and action of netrin-1. Generally, Mcl-1 is overexpressed and/or stabilized in these cancers. “Mcl-1+ tumor” means the tumor comprise tumor cells escaping programmed cell death or apoptosis by a mechanism involving Mcl-1 and netrin-1. In these tumors, Mcl-1 is expressed at a certain level which may be evaluated, and is enough to shift the balance between cellular survival and death towards the former. Also, methods to evaluate netrin-1 presence or levels are available to the skilled person. One may qualifies these tumors (or cancers) as “Mcl-1+/netrin-1+ tumors” or “Mcl-1+/netrin-1+/netrin-1 receptor+ tumors” or “Mcl-1+/Unc5B+ tumors”. “Mcl-1+/netrin-1+/Unc5B+ tumors”. In an aspect, the tumor is a Mcl1+ and netrin-1 receptor+ tumor (especially a receptor selected from UNC5A, B and C). In an aspect, the cancer or tumor further comprise the involvement of other anti-apoptotic Bcl-2 members (such as Bcl-2), which can be named herein “Bcl-2+”.

Mcl-1 level as well as other anti-apoptotic Bcl-2 members (such as Bcl-2) are measurable through classical immunohistochemical assays. Krajewska et al. (AJP May 1996, vol 148, No. 5:1567-1576) indicates that the generation and characterization of anti-peptide antisera specific for the human Bcl-2, Bax, Bcl-X, and Mcl-1 proteins as well as methods for the application of these antibodies for paraffin immunohistochemistry, are known and described in detail in articles. One may also refer to Erinna F. Lee et al. (CellDeath & Disease 2019, 10, 342). Mcl-1 level may be expressed different ways according to these and other authors: percentage of Mcl-1 positive tumor cells, or the immunostaining intensity. Any suitable method used by the clinicians in the hospitals are useful, as well.

For example, according to Erinna F. Lee et al., evaluation is made through antibody staining, cancer cells with positive staining in the cytoplasm are counted and the percentage of positive tumor cells graded (grades) as: 0: none; 1:1-5%; 2:6-25%; 3:26-50%; 4:51-75% and 5:76-100%. The intensity of staining can be rated as: 0: none; 1: weak; 2: moderate; 3: intense. An H-score for each sample can be calculated by multiplying the grading and intensity scores (range 0-15), then categorized as high (9-15) or low (as 0-8). In the present invention, a tumor may be qualified a “Mcl-1+ tumor” preferably when the percentage of positive tumor cells is equal or above grade 3. In some embodiments, tumor is qualified a “Mcl-1+ tumor” when the percentage of positive tumor cells is equal to or above grade 3, or equal to or above grade 4, or equal to grade 5.

Also, recent methods allow measuring protein amounts in biopsies using mass spectrometry, for example using the method described in Archer T et al., Cancer Cell. 2018 Sep. 10; 34 (3): 396-410 (PMID: 30205044). This may allow the skilled person getting Mcl-1 amounts in tumor biopsies, determining minimal amounts corresponding to “Mcl-1+ tumor”, and using the method to classify and select tumors for treatment.

In a further aspect, these cancers are expressing or overexpressing netrin-1 and the tumor cells present netrin-1 receptors, such as, in particular, UNC5B (UNC5B+, or “Mcl-1+/netrin-1+/UNC5B+ tumors”).

In a given aspect, the patient is first assayed to determine if he has a “Mcl-1+ tumor”, a “Mcl-1+/netrin-1+ tumors” or “Mcl-1+/netrin-1+/netrin-1 receptor+ tumors” or “Mcl-1+/netrin-1+/Unc5B+ tumors”. If he has such tumor, then he is treated with the anti-netrin-1 antibody. Thus, the different objects, i.e. use, for use, method of treatment, may comprise such an assay and/or determination of this patient status. In an embodiment, the assay is made (see Erinna F. Lee et al.) with antibody staining using any available antibody binding to Mcl-1, cancer cells with positive staining in the cytoplasm are counted and the percentage of positive tumor cells graded (grades) as: 0: none; 1:1-5%; 2:6-25%; 3:26-50%; 4:51-75% and 5:76-100%. In the present invention, a tumor may be qualified a “Mcl-1+ tumor” preferably when the percentage of positive tumor cells is equal or above grade 3. In some embodiments, tumor is qualified a “Mcl-1+ tumor” when the percentage of (Mcl-1) positive tumor cells is equal to or above grade 3, or equal to or above grade 4, or equal to grade 5.

Some embodiments of cancers concerned herein include metastatic breast cancer, non-small cell lung cancer, aggressive neuroblastoma, pancreatic adenocarcinoma, primary melanoma, melanoma metastasis, ovarian cancers, glioblastoma, acute myeloid leukaemia, chronic lymphocytic leukaemia, aggressive B-cell lymphoma, sarcoma, renal adenocarcinoma, head and neck cancers, testicular cancers (e.g. embryonal carcinoma, teratoma, yolk sac tumors), kidney cancers, stomach cancers, uterus cancers, colorectal cancer, lung adenocarcinoma.

Some specific embodiments of cancers exhibiting increased expression of netrin-1 include breast tumors, especially metastatic breast cancers, (Fitamant, 2008), lung cancer (Delloye-Bourgeois et al., 2009a), neuroblastoma (Delloye-Bourgeois et al., 2009b), lymphoma (Broutier et al., 2016), melanoma (Boussouar et al., 2020), inflammatory-associated colorectal cancer (Paradisi et al., 2008; Paradisi et al., 2009).

EP 3 092 003 provides an indication of the prevalence of netrin-1 overexpressing cancers in a number of cancers: 60% of metastatic breast cancer (Fitamant et al. 2008), 47% of non-small cell lung cancer (Delloye-Bourgeois et al. 2009a), 38% of aggressive neuroblastoma (Delloye-Bourgeois et al. 2009b), 61% of pancreatic adenocarcinoma (Link et al., Annals of Chir. Onco. 2007; Dumartin et al., Gastro 2010), 100% of primary melanoma (n=7), melanoma metastasis (n=6) (Kaufmann et al., Cellular Oncology 2009), 76% of ovarian cancers (Panastasiou et al., Oncotarget 2011), 65% of glioblastoma, 60% of acute myeloid leukemia and chronic lymphocytic leukemia, 50% of aggressive B-cell lymphoma, 30% of sarcoma, 40% of renal adenocarcinoma, 22% of head and neck cancers, Testicular cancers (36% of embryonal carcinoma, 50% of teratoma, 100% of yolk sac tumors), 50% of kidney cancers, 26% of stomach cancers, 19% of uterus cancers.

In such cancers overexpressing netrin-1, netrin-1 has been shown to behave as a survival factor for cancer cells (Mehlen et al., Nature cancer Rev 2011) but this survival has been proposed to be independent of the Bcl-2 proteins (Forcet et al. 2001, PNAS) in line with the view that the nomenclature of cell death mechanisms propose that dependence receptors such as the netrin-1 receptors triggers cell death independently of the mitochondrial dependent pathway (Galluzzi et al. 2018).

### Anti-NTN1 Antibodies (Anti-Netrin-1 Antibody or Antibody Binding to Netrin-1)

The antibody may be a polyclonal or monoclonal antibody specifically binding to netrin-1 (NTN1) (anti-netrin-1 antibody or antibody binding to netrin-1), especially human netrin-1. In an aspect, the antibodies used and disclosed herein are neutralizing. In an aspect, the anti-netrin-1 antibody or its netrin-1 binding fragment or derivative of this antibody is neutralizing. As used herein, the term “neutralizing antibody” refers to an antibody that captures netrin-1, blocks or reduces at least one activity of netrin-1, especially its binding to its receptors, primarily UNC5B.

An anti-netrin-1 polyclonal antibody may, inter alia, be obtained by immunizing an animal such as a rabbit, a mouse and the like with the aid of the selected amino acid sequence, collecting and then depleting the antiserum obtained on, for example, an immunoadsorbent containing the receptor according to methods known per se to a person skilled in the art. The netrin-1 amino acid sequence is as depicted on SEQ ID NO: 1 and netrin-1 may be used in whole or in part to generate polyclonal or monoclonal antibodies.

Generally, monoclonal antibodies may be obtained according to the conventional method of lymphocyte fusion and hybridoma culture described by Köhler and Milstein, (Nature, 1975, 256 (5517): 495-7). Other methods for preparing monoclonal antibodies are also known (Harlow et al., ed., 1988 “Antibodies: a laboratory manual”). The monoclonal antibodies may be prepared by immunizing a mammal (for example a mouse, a rat, a rabbit or even a human being, and the like) and using the lymphocyte fusion technique leading to hybridoma (Köhler and Milstein, 1975). Alternative techniques to this customary technique exist. It is possible, for example, to produce monoclonal antibodies by expressing a nucleic acid cloned from a hybridoma. It is also possible to produce antibodies by the phage display technique by introducing cDNAs for antibodies into vectors, which are typically filamentous phages which exhibit gene libraries V at the surface of the phage (for example fUSE5 for E. coli, Scott J. K., Smith G. P. Science 1990; 249:386-390). Protocols for constructing these antibody libraries are described in J. D. Marks et al., J. Mol. Biol., 222 (1991), p. 581). The cDNA corresponding to full length netrin-1 with signal sequence (SEQ ID NO: 2) or to a suitable fragment thereof may be used to produce monoclonal antibodies according to these methods.

The anti-netrin-1 monoclonal antibody (mAb) may be a murine, a chimeric, a humanized or a full-human monoclonal antibody. The fragment may be any type of mAb fragment that keeps substantially the ability of the whole antibody to bind to Netrin-1, it can be for example a Fab or a F (ab′) 2. In particular, a monoclonal antibody is one disclosed in WO2015/104360 or U.S. Pat. No. 10,494,427, which documents are incorporated herein by reference, and disclose useful murine, chimeric and humanized monoclonal antibodies. These are antibodies or fragments thereof, which specifically bind to a NTN1 epitope or polypeptide having the amino acid sequence SEQ ID NO: 3 or 33, or a variant thereof.

The monoclonal antibodies useful in the invention may be defined by their CDRs. In particular these CDRs are derived from the murine antibody 4C11 disclosed in WO2015/104360, which specifically binds to the polypeptide having the amino acid sequence SEQ ID NO: 3 or 33. Preferably, the antibody is a monoclonal antibody or an antigen-binding fragment thereof, comprising:
a variable domain VH comprising:
   a H-CDR1 having a sequence set forth as SEQ ID NO: 5;
   a H-CDR2 having a sequence set forth as SEQ ID NO: 6;
   a H-CDR3 having a sequence set forth as SEQ ID NO: 7;
a variable domain VL comprising:
   a L-CDR1 having a sequence set forth as SEQ ID NO: 8;
   a L-CDR2 having a sequence YAS;
   a L-CDR3 having a sequence set forth as SEQ ID NO: 9;
or
a variable domain VH comprising:
   a H-CDR1 having a sequence set forth as SEQ ID NO: 28;
   a H-CDR2 having a sequence set forth as SEQ ID NO: 29;
   a H-CDR3 having a sequence set forth as SEQ ID NO: 30;
a variable domain VL comprising:
   a L-CDR1 having a sequence set forth as SEQ ID NO: 31;
   a L-CDR2 having a sequence set forth as SEQ ID NO: 32;
   a L-CDR3 having a sequence set forth as SEQ ID NO: 9.

In a first series of embodiments, the antibody of the invention comprises an amino acid sequence SEQ ID NO: 10, 11, 12 or 13. Typically, it comprises a VH of sequence SEQ ID NO: 10 and a VL of sequence SEQ ID NO: 11, or a heavy chain of sequence SEQ ID NO: 12 and a light chain of sequence SEQ ID NO: 13.

In a second series of embodiments, the antibody is chimeric. Preferably, it comprises a VH of sequence SEQ ID NO: 27 and a VL of sequence SEQ ID NO: 19.

In a third series of embodiments, the antibody is humanized. Preferably, it comprises an amino acid sequence selected from the group of SEQ ID NO: 14 to 18 (VL) and/or from the group of SEQ ID NO: 20 to 26 (VH). Typically, the antibody is humanized and comprises a VH having an amino acid sequence selected from the group of SEQ ID NO: 14 to 18 and a VL having an amino acid sequence selected from the group of SEQ ID NO: 20 to 26.

The antibody preferably comprises a monoclonal antibody (mAb) or an antigen-binding fragment thereof, wherein the mAb or its fragment specifically binds to Netrin-1. The mAb may be a murine, a chimeric, a humanized or a full-human monoclonal antibody. The fragment may be any type of mAb fragment that keeps substantially the ability of the whole antibody to bind to Netrin-1, it can be for example a Fab or a F (ab′) 2.

Specific embodiments disclosed in this prior document and that can be used herein are the following antibodies listed in Table 1. The first listed antibody is a chimeric 4C11 antibody, comprising the murine VH and VL of the murine 4C11 antibody. HUM00 listed in Table 1 corresponds to the grafting of the murine 4C11 CDRs into a human IgG1. The ten humanized mAb HUM01 to HUM10 correspond to humanized mAbs derived from HUM00 with the same CDRs, but specific modifications in the FR regions of the human IgG. HUM03 is also publicly known as NP137 and is currently under clinical trials. Sequences of the human IgG1 CH come from Genbank AEL33691.1 modified R97K. Sequences of the human IgG1 CL (Kappa) come from Genbank CAC20459.1. The other allotypes may be used as well. Specific binding of all these mAbs, murine, chimeric and humanized HUM01-HUM10, Fab fragments and F(ab′) 2 fragments, to Netrin-1 and their ability to inhibit binding of netrin-1 to its receptor UNC5B, are demonstrated in U.S. Pat. No. 10,494,427 (Example 3).

In particular, these monoclonal antibodies specifically bind to the polypeptide having the amino acid sequence SEQ ID NO: 33.

TABLE 1

| | VH SEQ | Constant heavy | VL SEQ | Constant |
|---|---|---|---|---|
| | ID NO: | chain | ID NO: | light chain |
| 4C11 | 10 | Human IgG1 | 11 | Human IgG1 |
| HUM00 | 27 | Human IgG1 | 19 | Human IgG1 |
| HUM01 | 20 | Human IgG1 | 14 | Human IgG1 |
| HUM02 | 21 | Human IgG1 | 15 | Human IgG1 |
| HUM03 | 22 | Human IgG1 | 16 | Human IgG1 |
| HUM04 | 23 | Human IgG1 | 17 | Human IgG1 |
| HUM05 | 24 | Human IgG1 | 17 | Human IgG1 |
| HUM06 | 25 | Human IgG1 | 16 | Human IgG1 |
| HUM07 | 26 | Human IgG1 | 17 | Human IgG1 |
| HUM08 | 22 | Human IgG1 | 17 | Human IgG1 |
| HUM09 | 25 | Human IgG1 | 18 | Human IgG1 |
| HUM10 | 21 | Human IgG1 | 16 | Human IgG1 |

Preferably, the antibody is a monoclonal antibody or an antigen-binding fragment thereof, comprising a pair of VH and VL sequences selected from the following pairs: SEQ ID NO: 27 and 19, SEQ ID NO: 20 and 14, SEQ ID NO: 21 and 15, SEQ ID NO: 22 and 16, SEQ ID NO: 23 and 17, SEQ ID NO: 24 and 17, SEQ ID NO: 25 and 16, SEQ ID NO: 26 and 17, SEQ ID NO: 22 and 17, SEQ ID NO: 25 and 18, SEQ ID NO: 21 and 16. More preferably, the antibody is a monoclonal antibody or an antigen-binding fragment thereof, comprising a pair of VH and VL sequences SEQ ID NO: 22 and 16.

The anti-netrin-1 monoclonal antibody may further comprise a Human IgG1 Constant heavy chain (CH) and/or a Human IgG1 Constant light chain (CL), in particular a human kappa constant domain.

In an embodiment, sequences of the human IgG1 CH come from Genbank AEL33691.1 modified R97K. Sequences of the human IgG1 CL (Kappa) come from Genbank CAC20459.1. In an embodiment, the mAb is NP137 (AB_2811180 in The Antibody Registry) and comprises SEQ ID NO: 22 and 16 as VH, respectively VL sequences, and those specific IgG1 CH and CL.

The term “antigen-binding fragment” of a monoclonal antibody (or “antibody-binding portion”), as used herein, refers to one or more fragments of an antibody that retain the ability to specifically bind to netrin-1, especially to the same epitope or short peptide netrin-1 sequence (such as SEQ ID NO: 3 or 33) than the whole antibody. Especially, the fragment comprise the same VH and VL regions than the whole antibody. An antibody fragment may include, for example, a Fab fragment, a F(ab′)₂ fragment, a Fv fragment, a dAb fragment, a fragment containing a CDR, or an isolated CDR. In an aspect, the fragment comprises the VH and VL sequences of an antibody selected from HUM00 to HUM10.

As anti-netrin-1 monoclonal antibodies that may be used, one may cite other monoclonal antibodies, or their antigen-binding fragments, developed against human netrin-1 or against animal netrin-1, netrin-1 being very homologous among species. May be cited: Abcam antibodies ab126729, ab122903, ab201324, ab39370; AF1109, AF6419, AF128.

### BH3 Mimetics and B-Cell Lymphoma 2 (Bcl-2) Inhibitors:

A “Bcl-2 inhibitor” is a class of drug that functions by inhibiting anti-apoptotic B-cell lymphoma-2 (Bcl-2) protein, leading to programmed cell death of cells.

Venetoclax, also known in the art as GDC-0199, ABT-199, or RG7601, is a BCL-2 inhibitor. Venetoclax chemically is known as 4-[4-[[2-(4-chlorophenyl)-4,4-dimethylcyclohexen-1-yl]methyl]piperazin-1-yl]-N-[3-nitro-4-(oxan-4-ylmethylamino)phenyl] sulfonyl-2-(1H-pyrrolo[2,3-b]pyridin-5-yloxy)benzamide. Its developed structure is well-known. Venetoclax and processes for the preparation thereof are disclosed in U.S. Pat. No. 8,546,399. Venetoclax is marketed in the United States under the brand name VENCLEXTA™ by AbbVie, Inc., and is indicated for the treatment of chronic lymphocytic leukemia.

BH3 mimetics can be an inhibitor of Bcl-2 itself (Bcl-2 inhibitor) or an inhibitor of any other anti-apoptotic members of the Bcl-2 family. It can also be an inhibitor of at least two of these proteins, for example inhibitor of Bcl-2 and Bcl-XL, or of Bcl-2, Bcl-W and Bfl-1/A1, as it is the case of Navitoclax.

In some embodiments, the inhibitor is Venetoclax (ABT-199 or GDC-0199), ABT-737, BP 1002, SPC2996, APG-1252, obatoclax mesylate (GX15-070 MS), or PNT2258, Navitoclax.

### Definitions and Further Embodiments, Variants and Alternatives of the Invention

In the context of the invention, the term “treating” or “treatment”, as used herein, means reversing, alleviating, inhibiting the progress of, or preventing the disorder or condition to which such term applies, or one or more symptoms of such disorder or condition.

By the term “treating cancer” as used herein is meant in particular the inhibition of the growth of malignant cells of a tumour and/or the progression of metastases from said tumor. Such treatment can also lead to the regression of tumor growth, i.e., the decrease in size of a measurable tumor. In a particular embodiment, such treatment leads to a partial regression of the tumor or metastasis. In another particular embodiment, such treatment leads to the complete regression of the tumor or metastasis. In some aspect, treatment prevents metastasis. In the present invention, such treatment leads to cancer cell apoptosis or programmed cell death.

According to the invention, the term “patient” or “patient in need thereof” is intended for a human or non-human mammal affected or likely to be affected with a malignant tumor.

By a “therapeutically effective amount” is meant a sufficient amount of the active agents to treat said cancer disease, at a reasonable benefit/risk ratio applicable to any medical treatment. It will be understood, however, that the total daily usage of the active agents will be decided by the attending physician within the scope of sound medical judgment. The specific therapeutically effective dose level for any particular patient will depend upon a variety of factors including the disorder being treated and the severity of the disorder; activity of the specific polypeptide or antibody employed; the specific composition employed, the age, body weight, general health, sex and diet of the patient; the time of administration, route of administration, and rate of excretion of the specific active agents employed; the duration of the treatment; drugs used in combination or coincidental with the specific active agents employed; and like factors well known in the medical arts.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

### Pharmaceutical Compositions

The form of the pharmaceutical compositions including the polypeptide or antibody of the invention and the route of administration naturally depend upon the condition to be treated, the severity of the illness, the age, weight, and gender of the patient, etc.

The active agents of the invention can be formulated for a topical, oral, parenteral, intranasal, intravenous, intramuscular, subcutaneous or intraocular administration and the like. In a particular embodiment, the active agents of the invention are administered intravenously

In particular, the pharmaceutical compositions including the active agents of the invention may contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

To prepare pharmaceutical compositions, an effective amount of the active agents of the invention may be dissolved or dispersed in a pharmaceutically acceptable carrier or aqueous medium.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (for example, glycerol, propylene glycol, and liquid polyethylene glycol, and the like) and suitable mixtures thereof. The proper fluidity can be maintained, for example, by the use of a coating, such as lecithin, by the maintenance of the required particle size in the case of dispersion and by the use of surfactants, stabilizing agents, cryoprotectants or antioxidants. The prevention of the action of microorganisms can be brought about by antibacterial and antifungal agents. In many cases, it will be preferable to include isotonic agents, for example, sugars or sodium chloride.

Sterile injectable solutions are prepared by incorporating the active agents in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will determine the appropriate dose for the individual subject.

### Administration of Drugs and Method of Use:

As used herein, “simultaneously” is used to mean that the two agents are administered concurrently, whereas the term “in combination” is used to mean they are administered, if not simultaneously, then “sequentially” within a timeframe that they both are available to act therapeutically within the same time-frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction therebetween, and their respective half-lives.

In contrast to “in combination” or “sequentially”, “separately” is used herein to mean that the gap between administering one agent and the other is significant, i.e. several hours, and this may include the case wherein the first administered agent is no longer present in the bloodstream in a therapeutically effective amount when the second agent is administered. However, the first administered agent may still be present and active in the cancer cells.

In an embodiment of the invention, the anti-netrin-1 antibody or antigen-binding fragment thereof is administered sequentially or separately prior to the Bcl-2 inhibitor.

In a particularly preferred embodiment, the Bcl-2 inhibitor is administered sequentially or separately prior to the anti-netrin-1 antibody or antigen-binding fragment thereof.

In an embodiment, both the anti-netrin-1 antibody or antigen-binding fragment thereof and the Bcl-2 inhibitor are within the same composition with a pharmaceutically acceptable carrier, excipient and/or diluent.

In another embodiment, they are presented in separate pharmaceutical forms or kit-of-parts. This forms a composition or set or kit-of-parts comprising an anti-netrin-1 antibody or antigen-binding fragment thereof and a Bcl-2 inhibitor, for a simultaneous, separate or sequential administration to a patient. Thus the invention may comprise (i) a composition comprising the two active ingredients as a mixture, or (ii) a composition comprising those active ingredients kept separate in the same conditioning or in separate conditionings, each one of the active ingredients being in their own vial or containers, and one usually refer to the notion of a kit-of-parts in case (ii).

In one embodiment, the antibody or antigen-binding fragment or the simultaneously combination of the anti-netrin-1 antibody or antigen-binding fragment thereof and the Bcl-2 inhibitor thereof, is administered as a single dose, or an initial dose followed by administration of a second or a plurality of subsequent doses of the same, wherein the subsequent doses are separated by at least one day; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

In an embodiment of the method of treatment, use and compositions for use, the administration of the anti-netrin-1 antibody or antigen-binding fragment thereof and the Bcl-2 inhibitor is sequential or separate. The interval between both administrations may be at least 5, 10, 15, 20 or 24 hours, preferably between 24 and 96 hours, more preferably between 24 and 72 hours, or more, especially between 24 and 48 hours, for example 24 hours. In an embodiment, one agent or drug is simply administered the day after the administration of the other agent or drug. And, the first administered active ingredient (the anti-netrin-1 antibody or antigen-binding fragment thereof, or the Bcl-2 inhibitor), with respect to which said time interval is calculated, may be administered as a single dose, or an initial dose followed by administration of a second or a plurality of subsequent doses of the same, wherein the subsequent doses are separated by at least one day; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

The different pharmaceutical forms may be used in the methods of treatment of the invention, in sufficient amounts.

The invention does or may not imply a change of the dose regimen of the Bcl-2 inhibitor, such as Venetoclax. However, the synergy that occurs with the anti-netrin-1 antibody or antigen-binding fragment thereof may allow to using lower dose regimen of Bcl-2 inhibitor, such as Venetoclax, in a patient. The skill practitioner is able to determine the optimum dose regimen in the context of the combined treatment provided by the present invention.

The pharmaceutical compositions can be administered to a subject at a suitable dose, i.e. for the anti-netrin-1 antibody or antigen-binding fragment thereof at least 1 mg/kg body weight, e.g. about 1 mg/kg body weight to about 100 mg/kg body weight, in particular about 10 mg/kg body weight to about 60 mg/kg body weight of the subject in which cancer, is to be treated. The Bcl-2 inhibitor, such as Venetoclax, may be administered at the usual dose, or at a reduced dose with respect to the usual dose as far as the combination has a synergic efficacy. For example the dose of Bcl-2 inhibitor, such as Venetoclax, is reduced by 10, 20, 30, 40, 50%, or more.

As used herein, the term “synergistic” means that the active components, e.g. antibodies, produce a greater effect when used in combination than would be expected from adding the individual effects of the two components. Advantageously, a synergistic interaction may allow for lower doses of each component to be administered to a patient, thereby decreasing the toxicity of chemotherapy, whilst producing and/or maintaining the same therapeutic effect. Thus, in a particularly preferred embodiment, each component can be administered in a sub-therapeutic amount.

TABLE 2
Description of sequences:

| SEQ | | |
|---|---|---|
| ID | | |
| NO: | Description | Sequence |
| 1 | NTN1 amino acid (aa) | MMRAVWEALAALAAVACLVGAVRGGPGLSMFAGQAA |
| | sequence (seq.) with | QPDPCSDENGHPRRCIPDFVNAAFGKDVRVSSTCGRP |
| | signal peptide in bold | PARYCVVSERGEERLRSCHLCNASDPKKAHPPAFLTDL |
| | and linear epitope | NNPHNLTCWQSENYLQFPHNVTLTLSLGKKFEVTYVSL |
| | mapping in bold and | QFCSPRPESMAIYKSMDYGRTWVPFQFYSTQCRKMYN |
| | underlined | RPHRAPITKQNEQEAVCTDSHTDMRPLSGGLIAFSTLDG |
| | | RPSAHDFDNSPVLQDWVTATDIRVAFSRLHTFGDENED |
| | | DSELARDSYFYAVSDLQVGGRCKCNGHAARCVRDRDD |
| | | SLVCDCRHNTAGPECDRCKPFHYDRPWQRATAREANE |
| | | CVACNCNLHARRCRFNMELYKLSGRKSGGVCLNCRH |
| | | NTAGRHCHYCKEGYYRDMGKPITHRKACKACDCHPVG |
| | | AAGKTCNQTTGQCPCKDGVTGITCNRCAKGYQQSRSPI |
| | | APCIKIPVAPPTTAASSVEEPEDCDSYCKASKGKLKINMK |
| | | KYCKKDYAVQIHILKADKAGDWWKFTVNIISVYKQGTSRI |
| | | RRGDQSLWIRSRDIACKCPKIKPLKKYLLLGNAEDSPDQ |
| | | SGIVADKSSLVIQWRDTWARRLRKFQQREKKGKCKKA |
| 2 | NTN1 nucleic acid seq. | ATGATGCGCGCAGTGTGGGAGGCGCTGGCGGCGCT |
| | | GGCGGCGGTGGCGTGCCTGGTGGGCGCGGTGCGCG |
| | | GCGGGCCCGGGCTCAGCATGTTCGCGGGCCAGGCG |
| | | GCGCAGCCCGATCCCTGCTCGGACGAGAACGGCCAC |
| | | CCGCGCCGCTGCATCCCGGACTTTGTCAATGCGGCC |
| | | TTCGGCAAGGACGTGCGCGTGTCCAGCACCTGCGGC |
| | | CGGCCCCCGGCGCGCTACTGCGTGGTGAGCGAGCG |
| | | CGGCGAGGAGCGGCTGCGCTCGTGCCACCTCTGCAA |
| | | CGCGTCCGACCCCAAGAAGGCGCACCCGCCCGCCTT |
| | | CCTCACCGACCTCAACAACCCGCACAACCTGACGTG |
| | | CTGGCAGTCCGAGAACTACCTGCAGTTCCCGCACAA |
| | | CGTCACGCTCACACTGTCCCTCGGCAAGAAGTTCGAA |
| | | GTGACCTACGTGAGCCTGCAGTTCTGCTCGCCGCGG |
| | | CCCGAGTCCATGGCCATCTACAAGTCCATGGACTACG |
| | | GGCGCACGTGGGTGCCCTTCCAGTTCTACTCCACGC |
| | | AGTGCCGCAAGATGTACAACCGGCCGCACCGCGCGC |
| | | CCATCACCAAGCAGAACGAGCAGGAGGCCGTGTGCA |
| | | CCGACTCGCACACCGACATGCGCCCGCTCTCGGGCG |
| | | GCCTCATCGCCTTCAGCACGCTGGACGGGCGGCCCT |
| | | CGGCGCACGACTTCGACAACTCGCCCGTGCTGCAGG |
| | | ACTGGGTCACGGCCACAGACATCCGCGTGGCCTTCA |
| | | GCCGCCTGCACACGTTCGGCGACGAGAACGAGGAC |
| | | GACTCGGAGCTGGCGCGCGACTCGTACTTCTACGCG |
| | | GTGTCCGACCTGCAGGTGGGCGGCCGGTGCAAGTG |
| | | CAACGGCCACGCGGCCCGCTGCGTGCGCGACCGCG |
| | | ACGACAGCCTGGTGTGCGACTGCAGGCACAACACGG |
| | | CCGGCCCGGAGTGCGACCGCTGCAAGCCCTTCCACT |
| | | ACGACCGGCCCTGGCAGCGCGCCACAGCCCGCGAA |
| | | GCCAACGAGTGCGTGGCCTGTAACTGCAACCTGCAT |
| | | GCCCGGCGCTGCCGCTTCAACATGGAGCTCTACAAG |
| | | CTTTCGGGGCGCAAGAGCGGAGGTGTCTGCCTCAAC |
| | | TGTCGCCACAACACCGCCGGCCGCCACTGCCATTAC |
| | | TGCAAGGAGGGCTACTACCGCGACATGGGCAAGCCC |
| | | ATCACCCACCGGAAGGCCTGCAAAGCCTGTGATTGC |
| | | CACCCTGTGGGTGCTGCTGGCAAAACCTGCAACCAA |
| | | ACCACCGGCCAGTGTCCCTGCAAGGACGGCGTGACG |
| | | GGTATCACCTGCAACCGCTGCGCCAAAGGCTACCAG |
| | | CAGAGCCGCTCTCCCATCGCCCCCTGCATAAAGATC |
| | | CCTGTAGCGCCGCCGACGACTGCAGCCAGCAGCGTG |
| | | GAGGAGCCTGAAGACTGCGATTCCTACTGCAAGGCC |
| | | TCCAAGGGGAAGCTGAAGATTAACATGAAAAAGTACT |
| | | GCAAGAAGGACTATGCCGTCCAGATCCACATCCTGAA |
| | | GGCGGACAAGGCGGGGGACTGGTGGAAGTTCACGG |
| | | TGAACATCATCTCCGTGTATAAGCAGGGCACGAGCC |
| | | GCATCCGCCGCGGTGACCAGAGCCTGTGGATCCGCT |
| | | CGCGGGACATCGCCTGCAAGTGTCCCAAAATCAAGC |
| | | CCCTCAAGAAGTACCTGCTGCTGGGCAACGCGGAGG |
| | | ACTCTCCGGACCAGAGCGGCATCGTGGCCGATAAAA |
| | | GCAGCCTGGTGATCCAGTGGGGGGACACGTGGGCG |
| | | CGGCGGCTGCGCAAGTTCCAGCAGCGTGAGAAGAAG |
| | | GGCAAGTGCAAGAAGGCCTAGCG |
| 3 | NTN1 aa epitopic seq. | VACNCNLHARRCRFNMELYKLSGRKSGGVCLNCRHNT |
| | | AGRHCH |
| 4 | NTN1 epitopic cDNA | GTGGCCTGTAACTGCAACCTGCATGCCCGGCGCTGC |
| | seq. | CGCTTCAACATGGAGCTCTACAAGCTTTCGGGGCGC |
| | | AAGAGCGGAGGTGTCTGCCTCAACTGTCGCCACAAC |
| | | ACCGCCGGCCGCCACTGCCAT |
| 5 | aa seq. of CDR1-H | GYTFTSYN |
| | (IMGT) | |
| 6 | aa seq. of CDR2-H | IYPGNGDT |
| | (IMGT) | |
| 7 | aa seq. of CDR3-H | ARGGTGFAY |
| | (IMGT) | |
| 8 | aa seq. of CDR1-L | QSVSND |
| | (IMGT) | |
| — | aa seq. of CDR2-L | YAS |
| | (IMGT) | |
| 9 | aa seq. of CDR3-L | QQDYSSPWT |
| | (IMGT et Kabat) | |
| 10 | aa sequence of mouse | QAYLQQSGAELVRPGASVKMSCKASGYTFTSYNMHWV |
| | 4C11 VH | KQTPRQGLEWIGAIYPGNGDTSYNQKFKGKATLTVDKS |
| | | SSTAYMQLSSLTSEDSAVYFCARGGTGFAYWGQGTLV |
| | | TVSA |
| 11 | aa sequence of mouse | SIVMTQTPKFLLVSAGDRVTITCKASQSVSNDVAWYQQ |
| | 4C11 VL | KPGQSPKLLIYYASNRYTGVPDRFTGSGYGTDFTFTIST |
| | | VQAEDLAVYFCQQDYSSPWTFGGGTKLEIK |
| 12 | Full aa sequence of | QAYLQQSGAELVRPGASVKMSCKASGYTFTSYNMHWV |
| | 4C11 (VH + mouse | KQTPRQGLEWIGAIYPGNGDTSYNQKFKGKATLTVDKS |
| | IgG1 CH) | SSTAYMQLSSLTSEDSAVYFCARGGTGFAYWGQGTLV |
| | | TVSAAKTTPPSVYPLAPGSAAQTNSMVTLGCLVKGYFP |
| | | EPVTVTWNSGSLSSGVHTFPAVLESDLYTLSSSVTVPSS |
| | | PRPSETVTCNVAHPASSTKVDKKIVPRDCGCKPCICTVP |
| | | EVSSVFIFPPKPKDVLTITLTPKVTCVVVDISKDDPEVQF |
| | | SWFVDDVEVHTAQTQPREEQFNSTFRSVSELPIMHQD |
| | | WLNGKEFKCRVNSAAFPAPIEKTISKTKGRPKAPQVYTI |
| | | PPPKEQMAKDKVSLTCMITDFFPEDITVEWQWNGQPAE |
| | | NYKNTQPIMNTNGSYFVYSKLNVQKSNWEAGNTFTCSV |
| | | LHEGLHNHHTEKSLSHSPGK |
| 13 | Full aa sequence of | SIVMTQTPKFLLVSAGDRVTITCKASQSVSNDVAWYQQ |
| | 4C11 (VL + mouse | KPGQSPKLLIYYASNRYTGVPDRFTGSGYGTDFTFTIST |
| | Kappa CL) | VQAEDLAVYFCQQDYSSPWTFGGGTKLEIKRADAAPTV |
| | | SIFPPSSEQLTSGGASVVCFLNNFYPKDINVKWKIDGSE |
| | | RQNGVLNSWTDQDSKDSTYSMSSTLTLTKDEYERHNS |
| | | YTCEATHKTSTSPIVKSFNRNEC |
| 14 | VL aa sequence of | EIVMTQSPATLSVSPGERATLSCKASQSVSNDVAWYQQ |
| | humanized variant of | KPGKAPKLLIYYASNRYTGIPPRFSGSGYGTDFTLTINNI |
| | 4C11 | ESEDAAYYFCQQDYSSPWTFGQG |
| 15 | VL aa sequence of | DIQMTQSPSSLSASVGDRVTITCKASQSVSNDVAWFQQ |
| | humanized variant of | RPGQSPRRLIYYASNRYTGVPSRFSGSGSGTDFTFTISS |
| | 4C11 | LEAEDAATYYCQQDYSSPWTFGQG |
| 16 | VL aa sequence of | DIQMTQSPSSLSASVGDRVTITCKASQSVSNDVAWYQQ |
| | humanized variant of | KPGQAPRLLIYYASNRYTGIPPRFSGSGYGTDFTLTINNI |
| | 4C11 | ESEDAAYYFCQQDYSSPWTFGQG |
| 17 | VL aa sequence of | DIQMTQSPSSLSASVGDRVTITCKASQSVSNDVAWYLQ |
| | humanized variant of | KPGQSPQLLIYYASNRYTGVPSRFSGSGSGTDFTFTISS |
| | 4C11 | LEAEDAATYYCQQDYSSPWTFGQG |
| 18 | VL aa sequence of | DIVMTQTPLSLPVTPGEPASISCKASQSVSNDVAWYQQ |
| | humanized variant of | KPGQAPRLLIYYASNRYTGIPPRFSGSGYGTDFTLTINNI |
| | 4C11 | ESEDAAYYFCQQDYSSPWTFGQG |
| 19 | VL aa sequence of | EIVMTQSPATLSVSPGERATLSCRASQSVSNDVAWYQQ |
| | humanized variant of | KPGQAPRLLIYYASNRYTGIPARFSGSGSGTEFTLTISSL |
| | 4C11 | QSEDFAVYYCQQDYSSPWTFGQG |
| 20 | VH aa sequence of | QVQLVQSGAEVKKPGASVKVSCKASGYTFTSYNMHWV |
| | humanized variant of | RQATGQGLEWMGAIYPGNGDTSYNQKFKGRVTITADK |
| | 4C11 | STSTAYMELSSLRSEDTAVYYCARGGTGFAYWGQG |
| 21 | VH aa sequence of | QVQLQQSGPGLVKPSQTLSLTCAISGYTFTSYNMHWIR |
| | humanized variant of | QPPGKGLEWIGAIYPGNGDTSYNQKFKGRVTITADKSTS |
| | 4C11 | TAYMELSSLRSEDTAVYYCARGGTGFAYWGQG |
| 22 | VH aa sequence of | QVQLQQSGPGLVKPSQTLSLTCAISGYTFTSYNMHWVR |
| | humanized variant of | QATGQGLEWMGAIYPGNGDTSYNQKFKGRLTISKDTSK |
| | 4C11 | NQVVLTMTNMDPVDTATYYCARGGTGFAYWGQG |
| 23 | VH aa sequence of | EVQLVQSGAEVKKPGESLRISCKGSGYTFTSYNMHWV |
| | humanized variant of | RQATGQGLEWMGAIYPGNGDTSYNQKFKGRFTISRDD |
| | 4C11 | SKNTAYLQMNSLKTEDTAVYYCARGGTGFAYWGQG |
| 24 | VH aa sequence of | QVQLQESGPGLVKPSQTLSLTCTVSGYTFTSYNMHWV |
| | humanized variant of | RQAPGQGLEWMGAIYPGNGDTSYNQKFKGRVTISVDT |
| | 4C11 | SKNQFSLKLSSVTAADTAVYYCARGGTGFAYWGQG |
| 25 | VH aa sequence of | QVQLVQSGAEVKKPGASVKVSCKASGYTFTSYNMHWV |
| | humanized variant of | RQATGQGLEWMGAIYPGNGDTSYNQKFKGRVTITADK |
| | 4C11 | STSTAYMELSSLRSEDTAVYYCARGGTGFAYWGQG |
| 26 | VH aa sequence of | QVQLQQSGPGLVKPSQTLSLTCAISGYTFTSYNMHWVR |
| | humanized variant of | QATGQGLEWMGAIYPGNGDTSYNQKFKGRVTITADKST |
| | 4C11 | STAYMELSSLRSEDTAVYYCARGGTGFAYWGQG |
| 27 | VH aa sequence of | QVQLVQSGAEVKKPGASVKVSCKASGYTFTSYNMHWV |
| | humanized variant of | RQAPGQGLEWMGAIYPGNGDTSYAQKFKGRVTMTRDT |
| | 4C11 | STSTVYMELSSLRSEDTAVYYCARGGTGFAYWGQ |
| 28 | aa seq. of CDR1-H | SYNMH |
| | (Kabat) | |
| 29 | aa seq. of CDR2-H | AIYPGNGDTSYNQKFKG |
| | (Kabat) | |
| 30 | aa seq. of CDR3-H | GGTGFAY |
| | (Kabat) | |
| 31 | aa seq. of CDR1-L | KASQSVSNDVA |
| | (Kabat) | |
| 32 | aa seq. of CDR2-L | YASNRYT |
| | (Kabat) | |
| 33 | NTN1 aa epitopic seq. | ARRCRFNMELYKLSGRKSGGVC |
| 34 | NTN1 epitopic cDNA | GCCCGGCGCTGCCGCTTCAACATGGAGCTCTACAAG |
| | seq. | CTTTCGGGGCGCAAGAGCGGAGGTGTCTGC |

CDRs under IMGT are highlighted in bold in Table 1 where appropriate.

As used herein, a sequence “at least 85% identical to a reference sequence” is a sequence having, on its entire length, 85%, or more, in particular 90%, 91%, 92%, 93%, 94%, 95%, 96%, 97%, 98%, 99%, 99.5%, 99.6%, 99.7%, 99.8%, 99.9% or 100% sequence identity with the entire length of the reference sequence.

A percentage of “sequence identity” may be determined by comparing the two sequences, optimally aligned over a comparison window, wherein the portion of the polypeptide sequence in the comparison window may comprise additions or deletions (i.e. gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g. using the algorithm of Needleman and Wunsch (1970) J. Mol. Biol. 48:443. The percentage of sequence identity can be readily determined for instance using the program Needle, with the BLOSUM62 matrix, and the following parameters gap-open=10, gap-extend=0.5.

In the context of the invention, a “conservative amino acid substitution” is one in which an amino acid residue is substituted by another amino acid residue having a side chain group with similar chemical properties (e.g., charge or hydrophobicity). In general, a conservative amino acid substitution will not substantially change the functional properties of a protein. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine-tryptophan, lysine-arginine, alanine-valine, glutamate-aspartate, and asparagine-glutamine.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

An “antibody” may be a natural or conventional antibody in which two heavy chains are linked to each other by disulfide bonds and each heavy chain is linked to a light chain by a disulfide bond. There are two types of light chain, lambda (2) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site.

“Complementarity Determining Regions” or “CDRs” refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively.

As used herein, a “human framework region” is a framework region that is substantially identical (about 85%, or more, in particular 90%, 95%, 97%, 99% or 100%) to the framework region of a naturally occurring human antibody.

In the context of the invention, CDR/FR definition in an immunoglobulin light or heavy chain is to be determined based on Kabat or IMGT definitions.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. ((http://) www.bioinf.org.uk/abs/#cdrdef)

In the context of the invention, the amino acid residues of the antibody of the invention may be numbered according to the IMGT numbering system. The IMGT unique numbering has been defined to compare the variable domains whatever the antigen receptor, the chain type, or the species (Lefranc M.-P., “Immunology Today, 18, 509 (1997); Lefranc M.-P., The Immunologist, 7, 132-136 (1999).; Lefranc, M.-P. et al. Dev. Comp. Immunol., 27, 55-77 (2003).). In the IMGT unique numbering, the conserved amino acids always have the same position, for instance cysteine 23, tryptophan 41, hydrophobic amino acid 89, cysteine 104, phenylalanine or tryptophan 118. The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc. ((http://) www.imgt.org/IMGTScientificChart/Nomenclature/IMGT-FRCDRdefinition.html).

As used herein, the term “antibody” denotes conventional antibodies and fragments thereof, as well as single domain antibodies and fragments thereof, in particular variable heavy chain of single domain antibodies, and chimeric, humanized, bispecific or multispecific antibodies.

As used herein, antibody or immunoglobulin also includes “single_domain antibodies” which have been more recently described and which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered single domain antibodies. Single domain antibodies may be derived from any species including, but not limited to mouse, human, camel, llama, goat, rabbit and bovine. Single domain antibodies may be naturally occurring single domain antibodies known as heavy chain antibody devoid of light chains. In particular, Camelidae species, for example camel, dromedary, llama, alpaca and guanaco, produce heavy chain antibodies naturally devoid of light chain. Camelid heavy chain antibodies also lack the CH1 domain.

The variable heavy chain of these single domain antibodies devoid of light chains are known in the art as “VHH” or “nanobody”. Similar to conventional VH domains, VHHs contain four FRs and three CDRs. Nanobodies have advantages over conventional antibodies: they are about ten times smaller than IgG molecules, and as a consequence properly folded functional nanobodies can be produced by in vitro expression while achieving high yield. Furthermore, nanobodies are very stable, and resistant to the action of proteases. The properties and production of nanobodies have been reviewed by Harmsen and De Haard (2007) Appl. Microbiol. Biotechnol. 77:13-22.

The term “monoclonal antibody” or “mAb” as used herein refers to an antibody molecule of a single amino acid composition that is directed against a specific antigen, and is not to be construed as requiring production of the antibody by any particular method. A monoclonal antibody may be produced by a single clone of B cells or hybridoma, but may also be recombinant, i.e. produced by protein engineering.

“Fragments” of (conventional) antibodies comprise a portion of an intact antibody, in particular the antigen binding region or variable region of the intact antibody. Examples of antibody fragments include Fv, Fab, F(ab′)₂, Fab′, dsFv, (dsFv)₂, scFv, sc (Fv)₂, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 Da and antigen binding activity, in which about a half of the N-terminal side of H chain and the entire L chain, among fragments obtained by treating IgG with a protease, papain, are bound together through a disulfide bond.

The term “F(ab′) 2” refers to an antibody fragment having a molecular weight of about 100,000 Da and antigen binding activity, which is slightly larger than the Fab bound via a disulfide bond of the hinge region, among fragments obtained by treating IgG with a protease, pepsin.

A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, in particular by using gene recombination techniques. Divalent and multivalent antibody fragments can form either spontaneously by association of monovalent scFvs, or can be generated by coupling monovalent scFvs by a peptide linker, such as divalent sc (Fv)₂.

“dsFv” is a VH::VL heterodimer stabilized by a disulphide bond.

“(dsFv) 2” denotes two dsFv coupled by a peptide linker.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

In a particular embodiment, the epitope-binding fragment is selected from the group consisting of Fv, Fab, F(ab′)₂, Fab′, dsFv, (dsFv)₂, scFv, sc (Fv)₂, diabodies and VHH.

A “chimeric antibody”, as used herein, is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass.

The term “humanized antibody” refers to an antibody which is initially wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains. In an embodiment, a humanized antibody has constant domains of human origin. As used herein, the term “humanized antibody” refers to a chimeric antibody, which contain minimal sequence derived from non-human immunoglobulin, e.g. the CDRs.

The term “antibody” is used to encompass all these kinds of antibodies, fragments or combination thereof.

The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host.

Antibodies can be humanized using a variety of other techniques including CDR-grafting (EP0239400; WO91/09967; U.S. Pat. Nos. 5,530,101 and 5,585,089), veneering or resurfacing (EP0592106; EP0519596; Padlan (1991) Molecular Immunology 28 (4/5): 489-498; Studnicka et al. (1994) Protein Engineering 7 (6): 805-814; Roguska et al. (1994) Proc. Natl. Acad. Sci U.S.A. 91:969-973), and chain shuffling (U.S. Pat. No. 5,565,332). Human antibodies can be made by a variety of methods known in the art including phage display methods. See also U.S. Pat. Nos. 4,444,887, 4,716,111, 5,545,806, and 5,814,318; and International patent application WO98/46645, WO98/50433, WO98/24893, WO98/16654, WO96/34096, WO96/33735, and WO91/10741.

As used herein, the term “specificity” refers to the ability of an antibody to detectably bind an epitope presented on an antigen, such as netrin-1, while having relatively little detectable reactivity with non-netrin-1 proteins or structures (such as other proteins presented on cancer cells, or on other cell types). Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is netrin-1).

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

The invention will now be described using non-limiting examples referring to the figures.

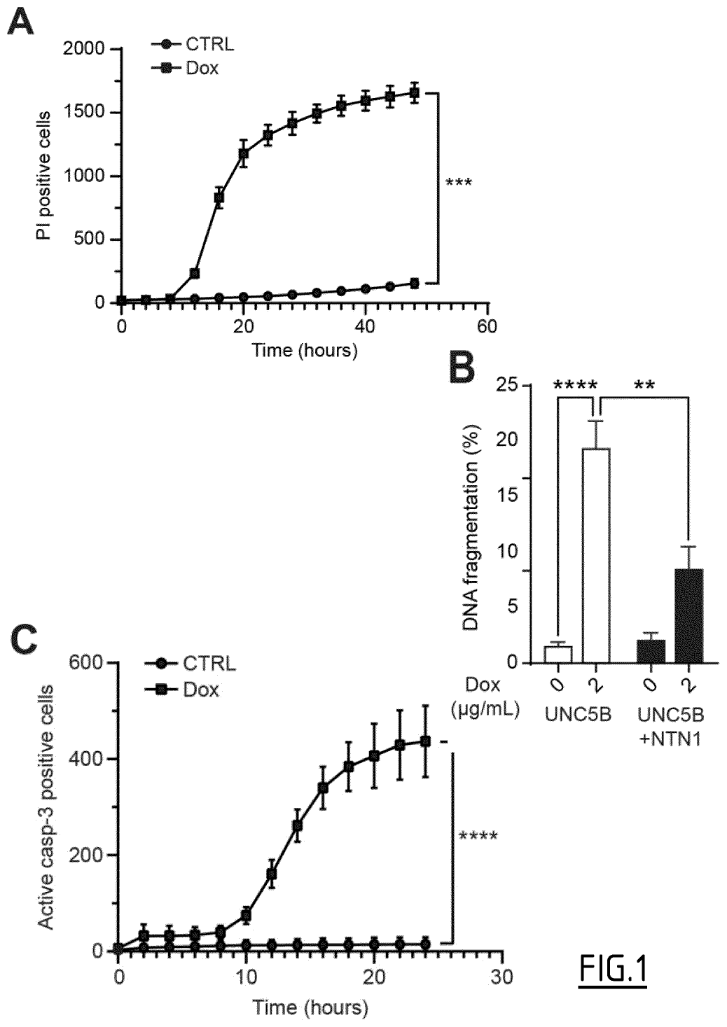

FIG. 1. CRISPR-Cas9 screening identifies genes and molecular pathways involved in UNC5B-induced cell death. (A): UNC5B over-expression triggers cell death. Stable Hela cells inducible for UNC5B (HeLa UNC5B) were treated with the indicated amount of doxycycline (Dox). To assess cell death, propidium iodide (PI) incorporation was followed in real-time up to 48 hours by Incucyte Zoom technology (n=3, *** p<0.01, Kolmogorov-Smirnov test). Values are given as means±SEM. (B,C): UNC5B-induced cell death is associated with DNA fragmentation and caspase-3 activation and is inhibited by netrin-1. HeLa UNC5B cells, constitutively expressing netrin-1 (UNC5B+NTN1) were treated with the indicated concentration of Dox for 18 hours and apoptosis was evaluated by DNA fragmentation assay through flow cytometry (n=3, ** p<0.01, **** p<0.0001, ordinary two-way ANOVA with Tukey's multiple comparisons test) (B). Caspase-3 activity was measured in real-time up to 24 hours using the Incucyte Zoom instrument (n=3, **** p<0.0001, Kolmogorov-Smirnov test) (C). Values are given as means±SEM.

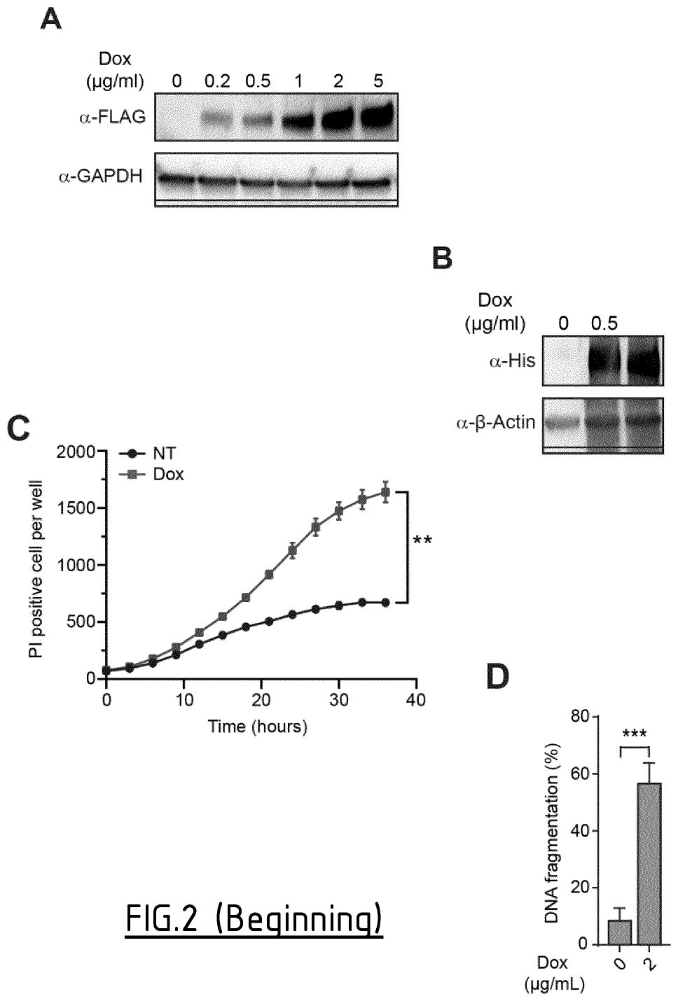

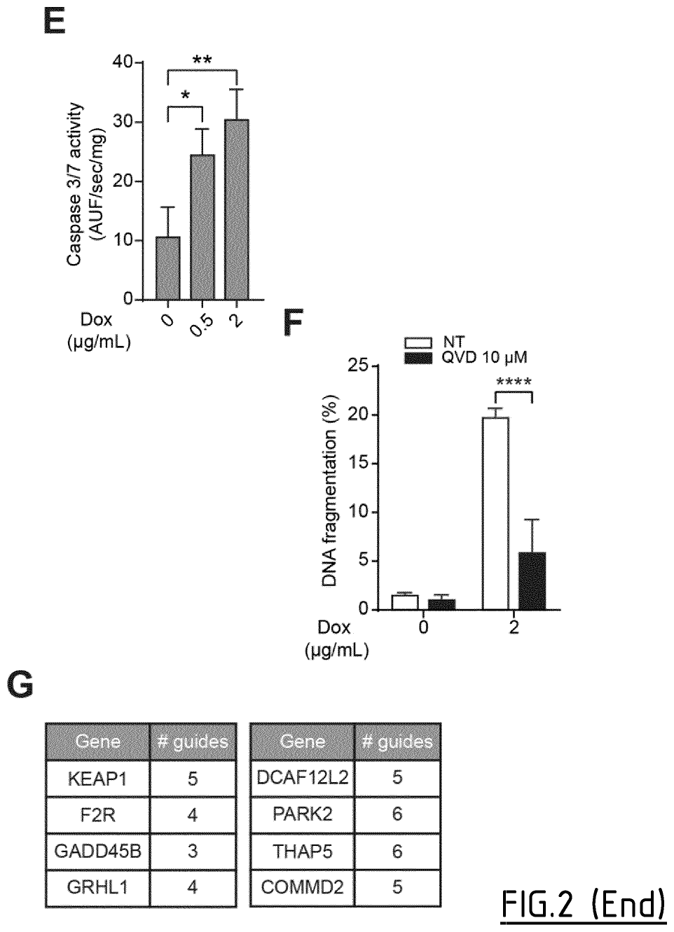

FIG. 2. CRISPR-Cas9 screening identifies genes and molecular pathways involved in UNC5B-induced cell death. Related to FIG. 1. (A): UNC5B induction upon treatment with doxycycline. Stable HCT116 cells, inducible for UNC5B, were treated with the indicated concentrations of doxycycline (Dox). 24 hours later, protein levels of FLAG-UNC5B and GAPDH, used as housekeeping gene, were assessed by western blot. (B): UNC5B induction upon treatment with doxycycline on Hela cells. Stable Hela cells inducible for UNC5B (HeLa UNC5B) were treated with the indicated doses of Dox for 18 hours. Protein levels of His-UNC5B and β-actin, used as housekeeping gene, were measured by western blot. (C,D,E): Induction of UNC5B triggers apoptotic cell death in HCT116 cells. Stable HCTT16 cells inducible for human UNC5B were treated with Dox. To assess cell death, propidium iodide (PI) incorporation was followed in real-time up to 36 hours, using an Incucyte Zoom technology (n=3, ** p<0.01, Kolmogorov Smirnov test) (C). Apoptosis was assessed 18 hours after Dox treatment by DNA fragmentation through flow cytometry (was evaluated 18 hours after Dox treatment by flow cytometry (D) and by caspase-3 activity (E), quantified by fluorometric detection kit (n=3, *p<0.05, ** p<0.01, *** p<0.001, Ordinary two-way ANOVA). Values are given as means±SEM. (F): Caspase inhibition rescues apoptosis triggered by UNC5B. HeLa UNC5B cells were treated with Dox and the pan-caspase inhibitor Q-VC-OPh (QVD). 18 hours later, DNA fragmentation was evaluated by flow cytometry (n=3, **** p<0.0001, ordinary two-way ANOVA with Šidák's multiple comparisons test). Values are given as means±SEM. (G): Candidate genes identified on CRISPR-Cas9-based screening. The number of guides showing an increase in reads compared to control condition is shown for each gene.

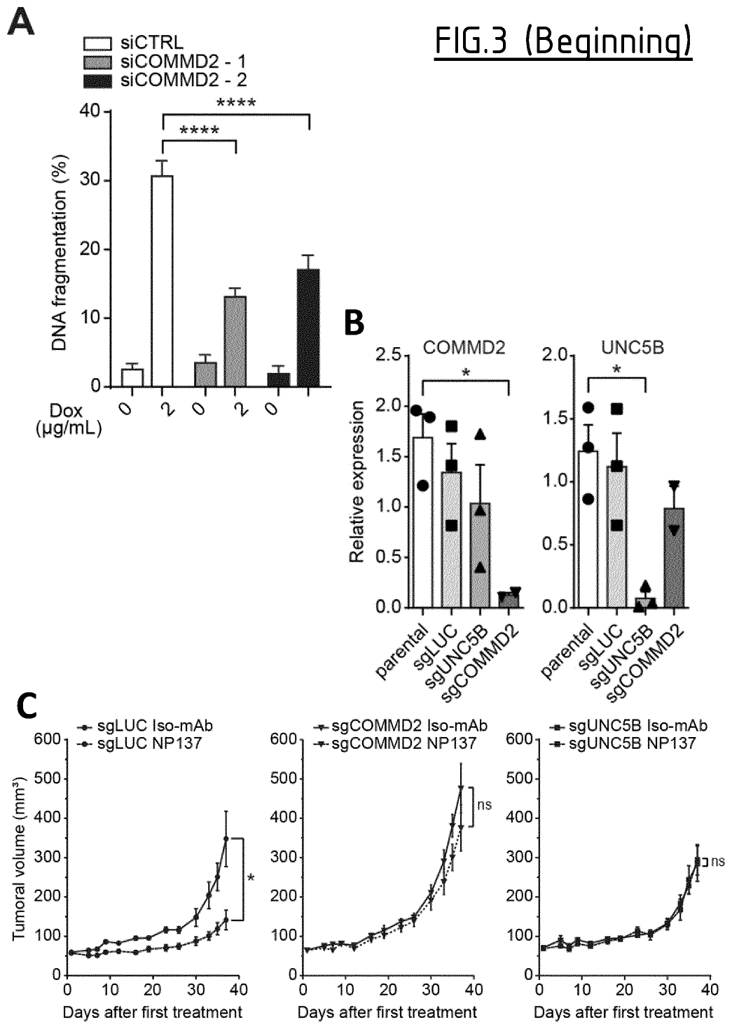

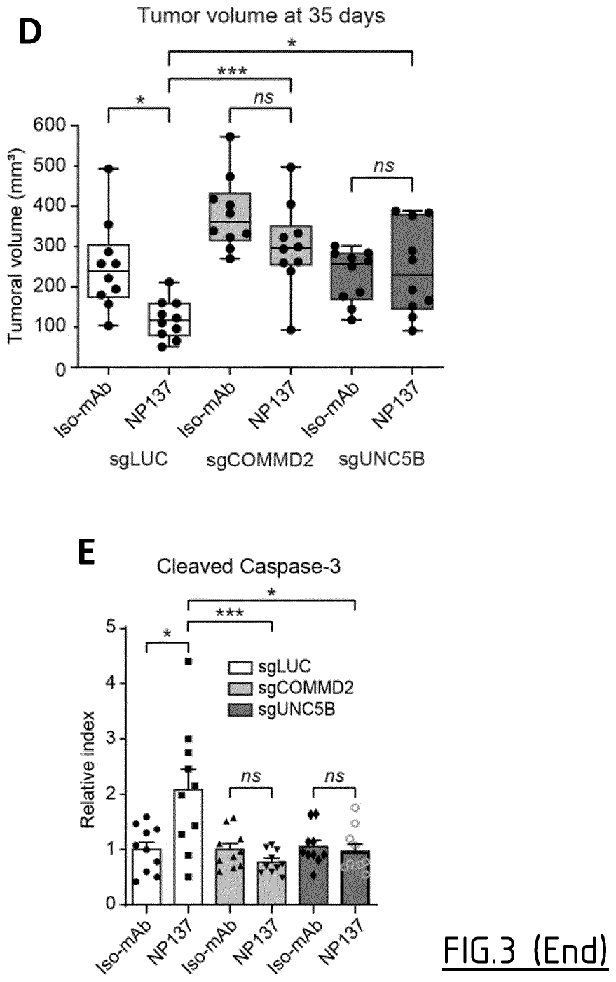

FIG. 3. COMMD2 is required for UNC5B-induced cell death and tumor suppression. (A): Silencing COMMD2 inhibits UNC5B-induced DNA fragmentation. Stable Hela cells inducible for UNC5B were transfected with two distinct siRNA scramble (siCTRL) or targeting COMMD2 (siCOMMD2-1 and -2). Upon treatment for 18 hours with the indicated concentrations of doxycycline (Dox), DNA fragmentation was evaluated through FACS analysis (n=3, **** p<0.0001, Ordinary two-way ANOVA). Values are given as means±SEM. (B, C, D): Tumor growth inhibition driven by netrin-1 interference necessitates UNC5B and COMMD2. NMRI nude mice were engrafted with stable H322 cells, knock-down for luciferase (sgLUC, used as a non-targeting control), UNC5B (sgUNC5B) or COMMD2 (sgCOMMD2), generated with CRISPR-Cas-9-KRAB strategy. Tumors obtained from nude mice engrafted with stable H322 cells were lysed and gene expression was evaluated by quantitative RT-PCR (n=2-3, *p<0.05, Kruskal-Wallis test with Dunn's multiple comparisons test) (B). After tumor volume reached 50 mm³, mice were intravenously treated three time per week with anti-netrin-1 (NP137) or control (Iso-mAb) antibodies and tumor growth was evaluated (n=10 mice/group, ^{ns}p>0.05, *p<0.05, Mann-Whitney test, two-tailed) (C). A histogram illustrating tumor volumes at 35 days after first treatment is indicated in (D) (n=10 mice/group, ^{ns}p>0.05, *p<0.05, *** p<0.001, Kruskal-Wallis test with uncorrected Dunn's test). (E). Silencing of UNC5B and COMMD2 restrains in vivo NP137-induced apoptosis. After sacrificing the mice, tumors were resected and apoptosis was quantified by immunostaining of active cleaved caspase-3. Positive cells were then indexed on tumors resected in Iso-mAb-treated mice for each stable cell line (n=10 mice/group, ^{ns}p>0.05, *p<0.05, *** p<0.001, Kruskal-Wallis test with uncorrected Dunn's test). Histogram represents means±SEM.

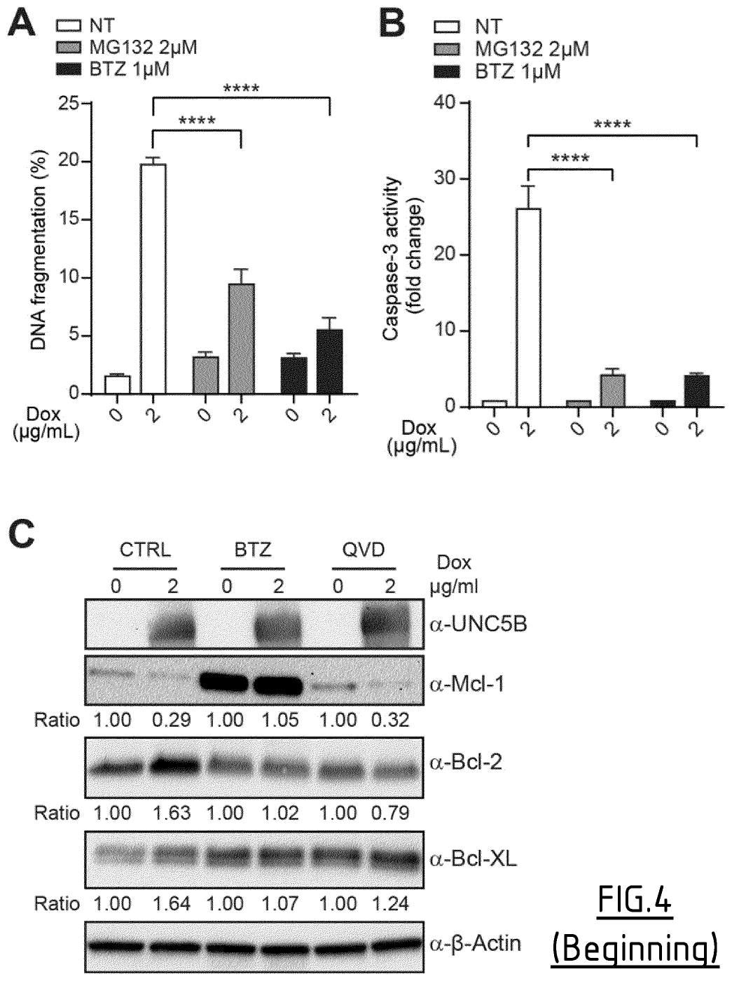

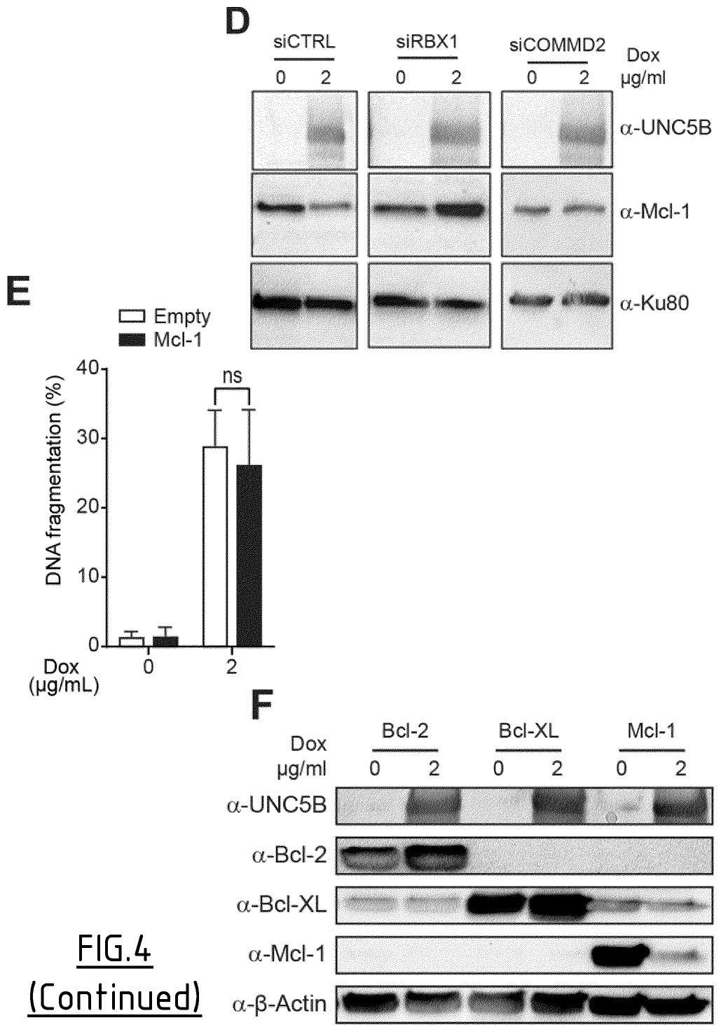

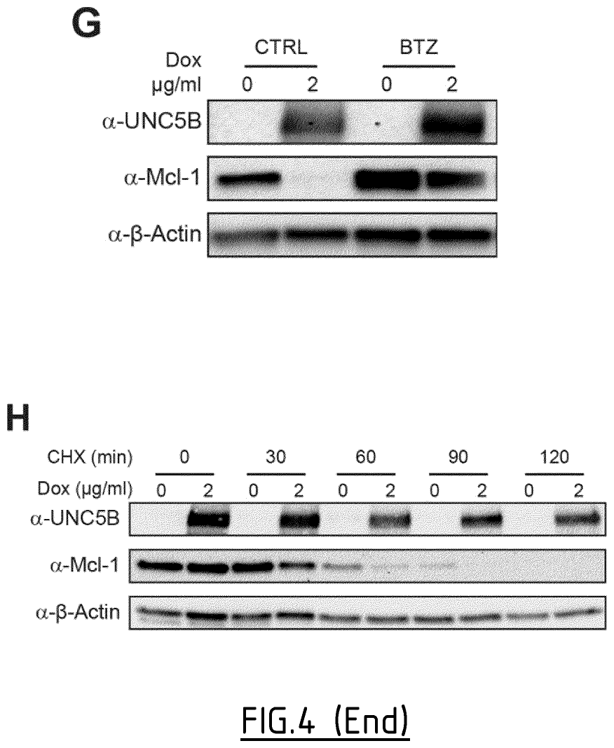

FIG. 4. UNC5B triggers proteasomal degradation of Mcl-1. (A,B): UNC5B-induced apoptosis requires proteasome activity. Stable Hela cells inducible for UNC5B (HeLa UNC5B) were treated with doxycycline (Dox) at 2 μg/mL, in combination with the indicated doses of the proteasome inhibitors MG132 and bortezomib (BTZ). 18 hours after treatment, apoptosis was measured by DNA fragmentation (A) and caspase-3 activity (B) (n=3-4, **** p<0.0001, ordinary two-way ANOVA with Tukey's multiple comparisons test). Values are given as means±SEM Treatment with proteasome inhibitors was sufficient to strongly reduce apoptosis induction upon UNC5B over-expression. (C): UNC5B triggers Mcl-1 degradation. HeLa UNC5B cells were treated with 2 μg/mL of Dox, in combination with bortezomib (BTZ, 1 μM) and the pan-caspase inhibitor Q-VD-OPh (QVD, 10 UM) inhibitors. 16 hours after treatment, cells were lysed by scraping and sonication in SDS-containing buffer. Protein levels of Mcl-1, Bcl-2 and Bcl-XL, as well as of UNC5B and B-actin, used as housekeeping gene, were evaluated by western blot. Ratio of protein levels between not-treated (0) and doxycycline-treated (2), normalized to B-actin, is showed in the bottom of each western blot. UNC5B over-expression specifically promoted Mcl-1 protein decrease. Proteasome inhibition, via treatment with bortezomib, rescued this degradation, while caspase inhibition through Q-VD-OPh, even though inhibited cell death, did not affect Mcl-1 degradation. (D): Mcl-1 degradation induced by UNC5B requires COMMD2/CRL3 complex. HeLa UNC5B cells were transfected with scramble (siCTRL), COMMD2 (siCOMMD2) or RBX1 (siRBX1) siRNAs. 24 hours after transfection, cells were treated with 2 μg/mL of Dox for further 18 hours. Protein levels of Mcl-1, UNC5B and Ku80, used as housekeeping protein, were evaluated by western blot. Silencing COMMD2 or RBX1 was sufficient to block Mcl-1 degradation elicited by UNC5B over-expression. (E): Mcl-1 over-expression is not sufficient to rescue UNC5B-induced apoptosis. Stable HeLa UNC5B cells, constitutively expressing Mcl-1 or empty vector, were treated with doxycycline and apoptosis was evaluated 18 hours later by DNA fragmentation (n=3, ^{ns}p>0.05, ordinary two-way ANOVA with Sidak's multiple comparisons test). Values are given as means±SEM. (F): Over-expressed Mcl-1 protein, but not Bcl-2 or Bcl-XL, is strongly degraded upon UNC5B induction. Stable HeLa UNC5B cells, constitutively expressing Mcl-1, Bcl-2 or Bcl-XL were treated with Dox and protein levels were evaluated as described in (C). (G): Proteasome activity is required for over-expressed Mcl-1 degradation upon UNC5B induction. Stable HeLa UNC5B cells, constitutively expressing Mcl-1, were treated with 2 μg/mL of Dox, in combination with the proteasome inhibitor bortezomib (BTZ, 1 M). 18 hours later, protein levels of Mcl-1, UNC5B and β-actin, used as housekeeping gene, were evaluated by western blot. (H): UNC5B speeds up the degradation of Mcl-1, lowering the half-life. HeLa UNC5B cells, constitutively expressing Mcl-1, were treated with Dox for six hours, and then Cycloheximide (CHX) was added. Cells were harvested at the indicated times after CHX treatment, and protein levels were assessed by western blot. Mcl-1 expression was normalized with B-actin. In the presence of UNC5B, Mcl-1 half-life decreased compared with the control condition.

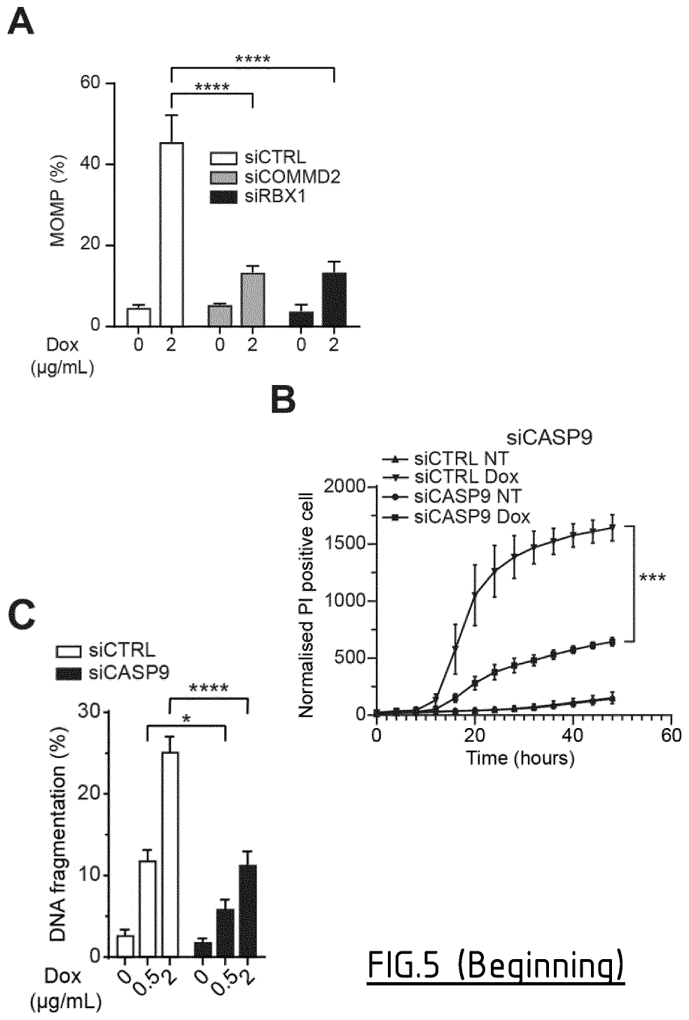

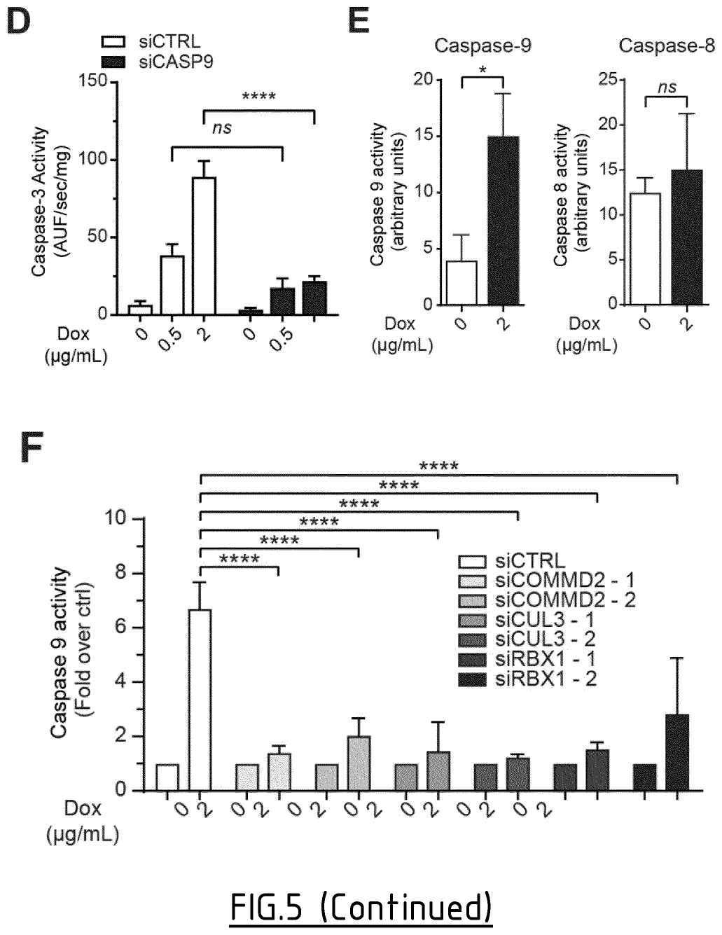

FIG. 5. UNC5B-mediated Mcl-1 degradation induces MOMP and caspase-9 activation. (A): UNC5B over-expression triggers Mitochondrial Outer Membrane Permeabilization (MOMP) in a COMMD2/CRL3 complex-dependent manner. Hela cells were transfected with scramble (siCTRL), COMMD2 (siCOMMD2-1) or RBX1 (siRBX1-1) siRNAs. 24 hours after transfection, cells were treated with Dox and after further 18 hours mitochondrial depolarization quantified by 5′,6,6′-tetrachloro-1,1′,3,3′-tetraethylbenzimi-dazolylcarbocyanine iodide (JC-1) dye staining, using NucleoCounter NC3000 (n=3, **** p<0.0001, ordinary two-way ANOVA with Tukey's multiple comparisons test). Values are given as means±SEM. (B,C,D): Apoptotic cell death induced by UNC5B over-expression requires caspase-9. HeLa UNC5B cells were transfected with scramble (siCTRL) or caspase-9 (siCASP9) siRNAs and 24 hours later treated with the indicated concentrations of Dox. Propidium iodide (PI)-positive cells were assessed in real-time up to 48 hours using Incucyte Zoom instrument (C) (n=3, *** p<0.01, Kolmogorov-Smirnov test). DNA fragmentation was assessed by FACS analysis (D) and caspase-3 was measured using caspase-3 fluorometric kit (E), both 18 hours after treatment (n=3, *p>0.05, **** p<0.0001, ordinary two-way ANOVA with Šidák's multiple comparisons test). Values are given as means±SEM. Inhibition of caspase-9 gene expression decreased cell death triggered by UNC5B upon Dox treatment. (E): UNC5B over-expression activates caspase-9, but not caspase-8. HeLa UNC5B cells were treated with Dox and 18 hours later caspase-9 and -8 activities were assessed by fluorometric kit, using specific substrates (n=3, ^{ns}p>0.05, *p<0.05, Mann-Whitney test, one-tailed). Values are given as means±SEM. UNC5B triggered a strong induction of caspase-9 activity, whereas caspase-8 activity did not increase after Dox treatment. (F): UNC5B-induced caspase-9 activity requires COMMD2/CRL3 complex. HeLa UNC5B cells were transfected with scramble siRNA (siCTRL), or siRNAs targeting COMMD2 (siCOMMD2-1 and -2), Cullin-3 (siCUL3-1 and -2), or RBX1 (siRBX1-1 and -2). 24 hours later, cells were treated with Dox and caspase-9 activity was measured by fluorometric kit (n=3, **** p<0.0001, ordinary two-way ANOVA with Tukey's multiple comparisons test). Values are given as means±SEM. Silencing of at least one member of COMMD2/CRL3 complex was adequate to suppress caspase-9 activity induced by UNC5B. (G): Proteasome or NEDD8 inhibition limits caspase-9 activity triggered by UNC5B. HeLa UNC5B cells were treated with Dox and the proteasome inhibitors MG132 and bortezomib (BTZ), the NEDD8 inhibitor pevonedistat or the pan-caspase inhibitor Q-VD-OPh (QVD), at the indicated concentrations. 18 hours after treatment, caspase-9 activity was assessed with the fluorometric kit (n=5, *p<0.05, ** p<0.01, *** p<0.001, **** p<0.0001, ordinary two-way ANOVA with Tukey's multiple comparisons test). Values are given as means±SEM. (H): Caspase-9 is not required for MOMP triggered by UNC5B. Hela UNC5B cells were transfected with scramble or caspase-9 specific siRNAs. 24 hours later, cells were treated with Dox, and after another 18 hours, mitochondrial depolarization rate was quantified as described in (B) (n=3, ^{ns}p<0.05, ** p<0.01, *** p<0.001, ordinary two-way ANOVA with Tukey's multiple comparisons test). Values are given as means±SEM. (I): Caspase-9 is not required for UNC5B-dependent Mcl-1 degradation. HeLa UNC5B cells were transfected with scramble or caspase-9 siRNAs. Cells were then treated with Dox and Mcl-1 protein was evaluated by western blot. Silencing of caspase-9 was not sufficient to revert Mcl-1 degradation observed upon UNC5B induction. (J): UNC5B-induced apoptosis involves the pro-apoptotic proteins BAX and BAK1. HeLa UNC5B cells were transfected with siRNAs targeting BAK1 and BAX, alone or combined. 24 hours later, cells were treated with Dox and after further 18 hours DNA fragmentation was evaluated by flow cytometry (n=4, **** p<0.0001, ordinary two-way ANOVA with Tukey's multiple comparisons test). Values are given as means±SEM.

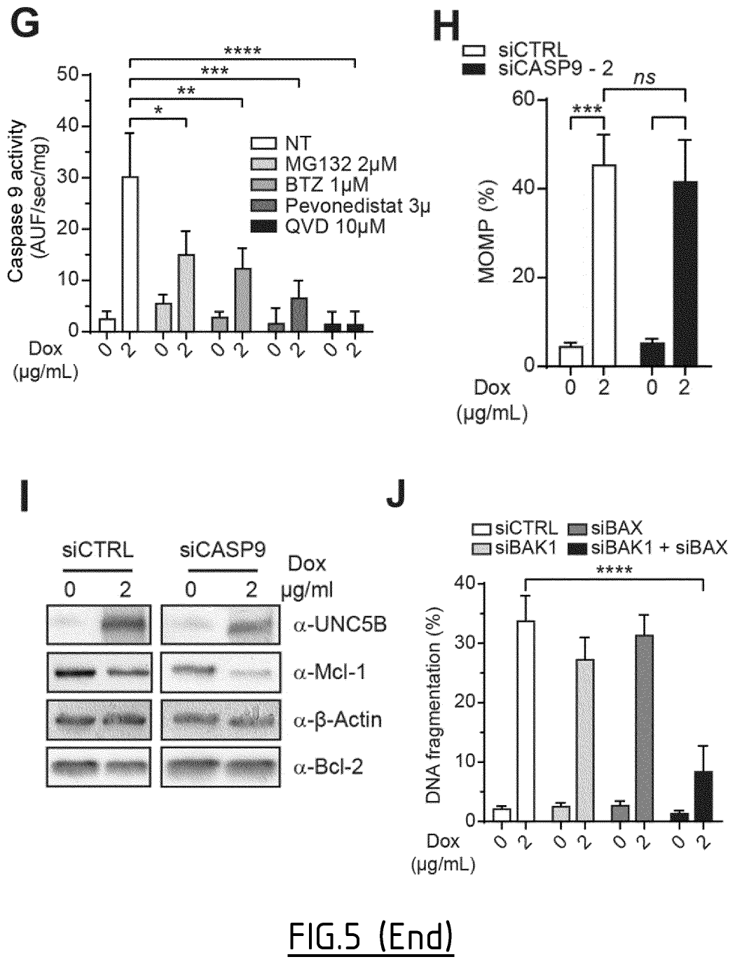

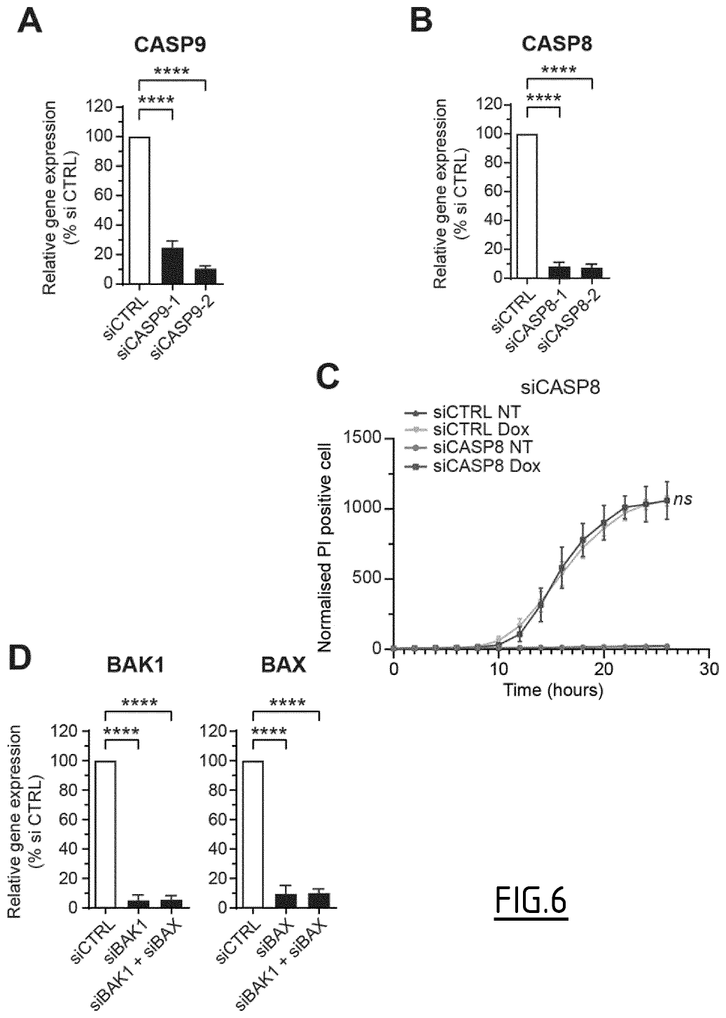

FIG. 6. UNC5B-mediated Mcl-1 degradation induces MOMP and caspase-9 activation. (A,B): Validation of caspase-9 and -8 silencing. HeLa UNC5B cells were transfected with scramble (siCTRL), caspase-9 (siCASP9-1 and -2) or caspase-8 (siCASP8-1 and -2) siRNAs. 24 hours after transfection, total RNA was extracted and caspase-9 (A) and -8 (B) gene expression was assessed by quantitative RT-PCR. Relative gene expression was normalized to siCTRL condition, using TATA binding protein (TBP) and β-glucuronidase (GUSB) genes as housekeeping genes (n=3-4, **** p<0.0001, ordinary two-way ANOVA with Dunnett's multiple comparisons test). Values are given as means±SEM. (C): Caspase-8 is not required for UNC5B-induced cell death. HeLa UNC5B cells were transfected with scramble (siCTRL) or caspase-8 (siCASP8) siRNAs. 24 hours after transfection, cells were treated with doxycycline (Dox) and cell death was assessed by measuring propidium iodide (PI) positive cells in real-time up to 26 hours, using Incucyte Zoom instrument (n=3, ^{ns}p>0.05, Kolmogorov-Smirnov test). Values are given as means±SEM. (D): Validation of BAX and BAK1 silencing. HeLa UNC5B cells were transfected using scramble (siCTRL), BAX (siBAX) or BAK1 (siBAK1) siRNAs. Quantitative RT-PCR was performed as in (A) (n=3, **** p<0.0001, ordinary two-way ANOVA with Dunnett's multiple comparisons test). Values are given as means±SEM.

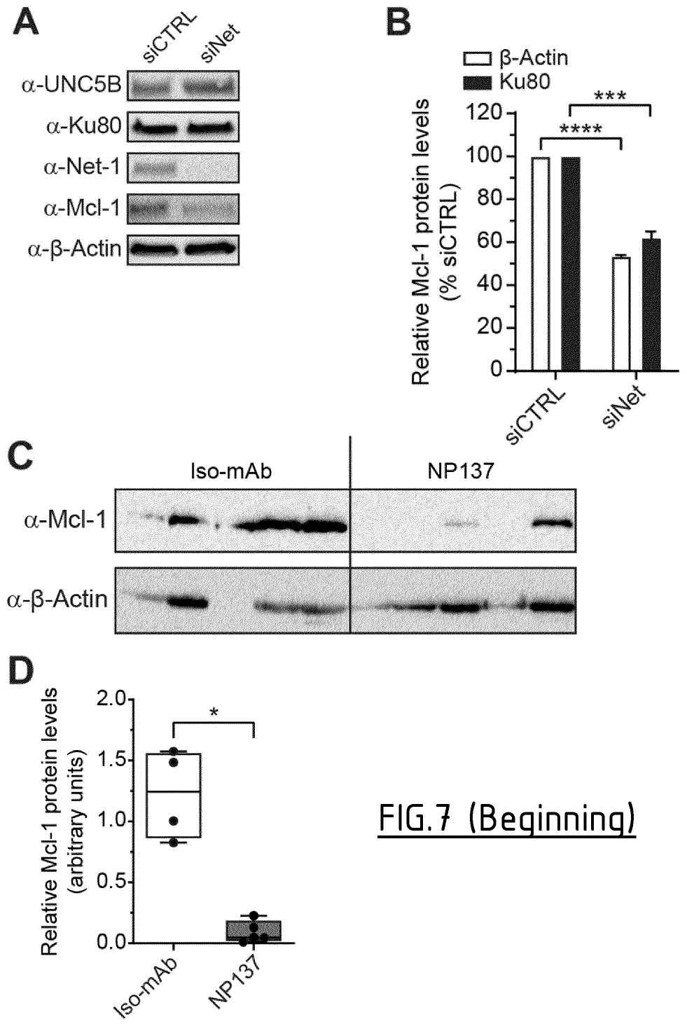

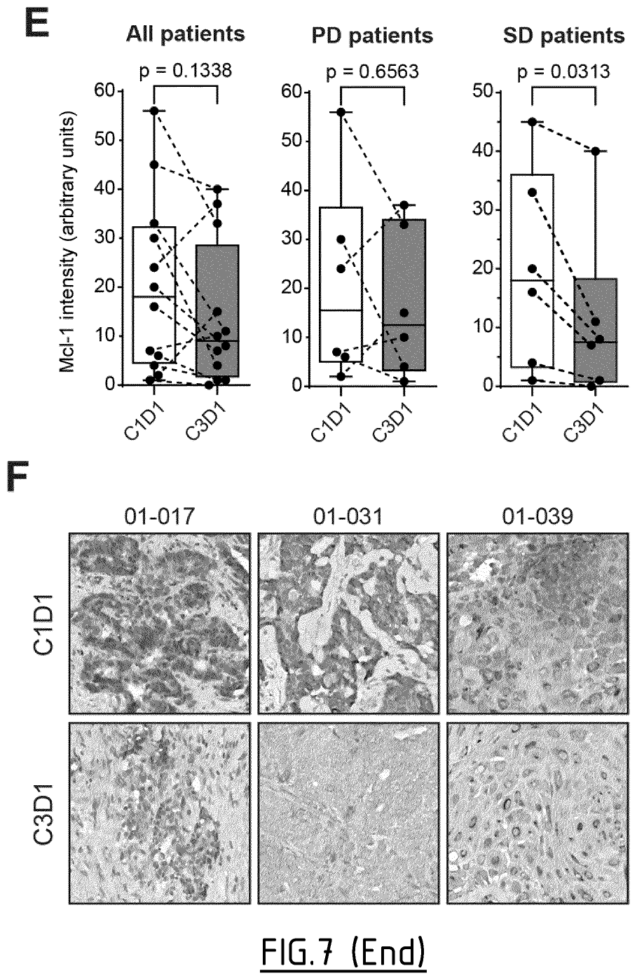

FIG. 7. Netrin-1 interference triggers Mcl-1 degradation in vitro and in vivo. (A,B): Netrin-1 silencing triggers Mcl-1 degradation. Breast cancer MCF7 cells were transfected with scramble (siCTRL) or netrin-1 (siNet) targeting siRNAs. 48 hours after transfection, cells were lysed by scraping and sonication in SDS-containing buffer. Protein levels of Mcl-1, netrin-1, and UNC5B, as well as B-actin and Ku80, used as housekeeping genes, were evaluated by western blot (A). Relative Mcl-1 protein levels, normalized to β-actin or Ku80, are shown in (B) (n=2, *** p<0.001, **** p<0.0001, ordinary two-way ANOVA with Sidak's multiple comparisons test). Activation of endogenous UNC5B upon netrin-1 silencing decreased Mcl-1 protein levels. Values are given as means±SEM. (C,D): Netrin-1 interference degrades Mcl-1 in a syngeneic breast cancer model. Stable EMT6 cells, constitutively over-expressing human Mcl-1, were engrafted in BALB/c mice. Once tumors reached a volume of 100 mm³, mice were treated with Iso-mAb or NP137 antibodies and sacrificed 8 hours after treatment. Protein levels of Mcl-1 were then evaluated by western blot (C). Relative Mcl-1 levels, normalized to β-actin, are shown in (D) (n=4-5, *p<0.01, Mann-Whitney test). The line in the middle of the boxes is plotted at the median. (E,F): NP137 antibody triggers Mcl-1 degradation in a cohort of cancer patients. Patients with advanced solid cancer, enrolled in phase I clinical trial (NCT02977195), were treated with 14 mg/kg of NP137 once every two weeks, according to the design of the phase I trial. Biopsies were collected at patient inclusion (before the first dose of NP137) (C1D1) and before the third infusion of NP137 (C3D1). Mcl-1 protein was quantified in paraffin-embedded slices on paired C1D1 and C3D1 biopsies and compared with clinical benefit (stable disease, SD, or progression disease, PD, at 6 weeks). Mcl-1 intensity was assessed in all, PD or SD biopsies (E) (n=6, Wilcoxon test). The dotted lines represent Mcl-1 expression in the same patient before and after NP137 infusion. A representative IHC images for three SD patients are shown in (F).

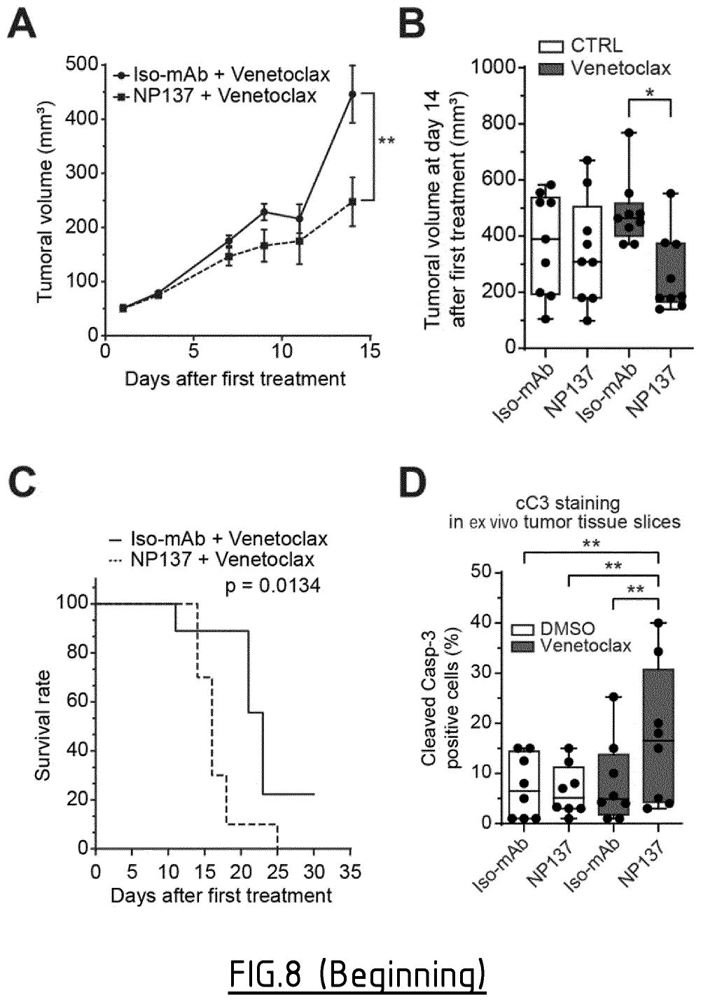

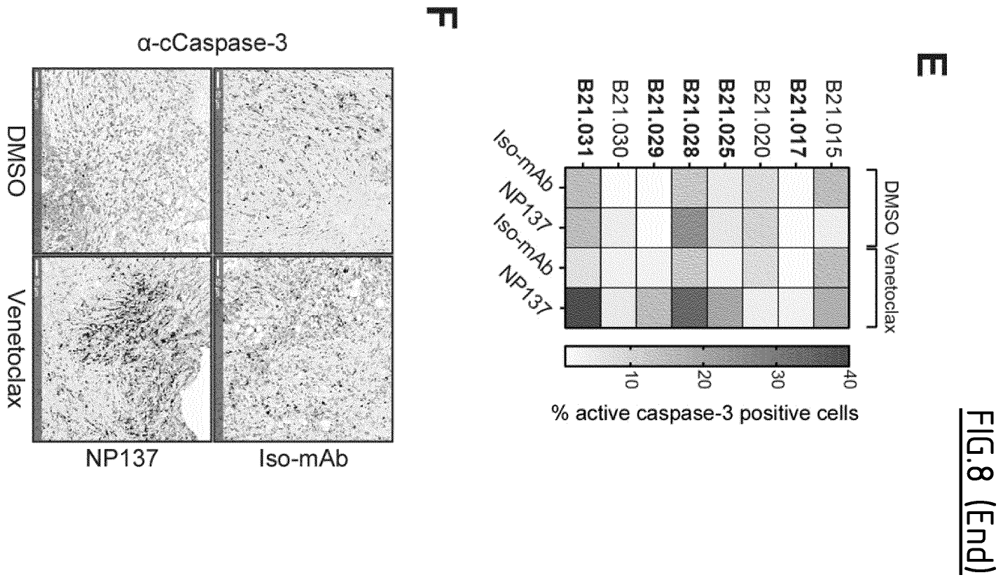

FIG. 8. Combining netrin-1 interference and BH3 mimetics inhibits tumor growth in preclinical in vivo and ex vivo models. ((A,B,C): Combining netrin-1 interference and Bcl-2 inhibition restrains tumor growth in a syngeneic breast cancer model. EMT6 cells were engrafted in BALB/c mice and, once tumors reached a volume of 50 mm³, treated with isotypic (Iso-mAb) or NP137 antibodies and the Bcl-2 inhibitor Venetoclax, as single agents or in combination. Tumor volumes were measured at the indicated days after the first treatment (n=9, ** p<0.01, ordinary two-way ANOVA with Tukey's multiple comparisons test) (A,B). Survival rate was assessed at sacrifice of mice with tumors more than 500 mm³ (C). (D,E,F): Combined treatment is efficient to induce apoptosis in human ex vivo slices. Cleaved active caspase-3-positive cells were assessed by IHC staining in fresh ex vivo breast tumor tissue slices. Combined treatment with NP137 antibody and Venetoclax increased apoptosis rate, compared to single agent treatments (n=8, ** p<0.01, ordinary two-way ANOVA with uncorrected Fisher's LSD).

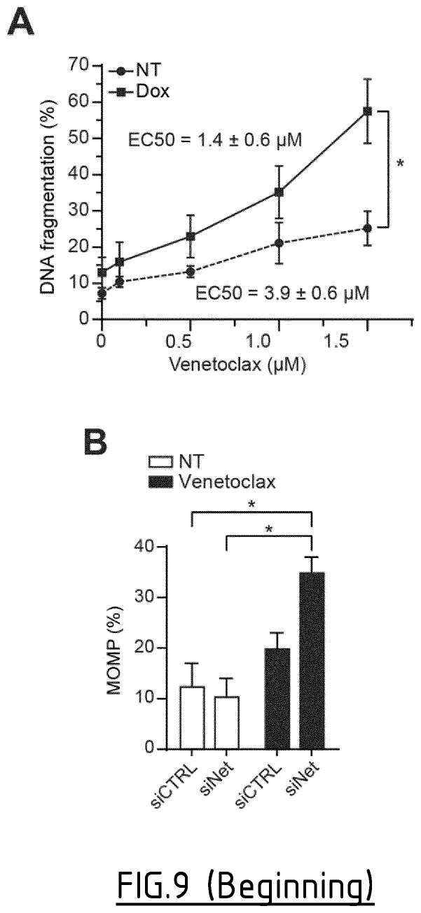

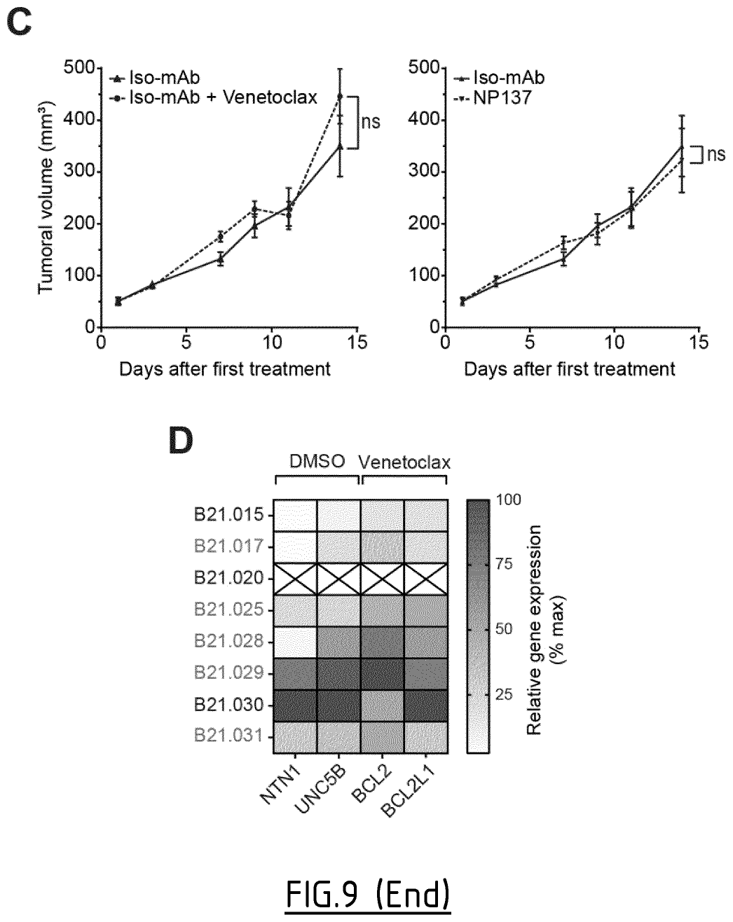

FIG. 9. Combining netrin-1 interference and BH3 mimetics inhibits tumor growth in preclinical in vivo and ex vivo models. Related to FIG. 8. (A): UNC5B over-expression sensitizes tumor cells to Bcl-2 inhibition. Stable breast cancer T47D cells, inducible for UNC5B, were treated with 2 μg/mL of doxycycline (Dox) and the indicated concentrations of the Bcl-2 inhibitor Venetoclax. 24 hours later, apoptosis was quantified by Annexin V/propidium iodide (PI) staining. EC50 values were calculated using nonlinear regression of Dose-response curves with GraphPad Prism software. (B): Silencing of netrin-1 gene expression increases cell death induced by Bcl-2 inhibition. Breast cancer MCF7 cells were transfected with scramble (siCTRL) or netrin-1 (siNet) targeting siRNAs. 24 hours later after transfection, cells were treated with Venetoclax for further 48 hours. Apoptosis was then evaluated by 5′,6,6′-tetrachloro-1,1′,3,3′-tetraethylbenzimi-dazolylcarbocyanine iodide (JC-1) dye staining, using NucleoCounter NC3000 (n=2, *p<0.05, ordinary two-way ANOVA with Tukey's multiple comparisons test). Values are given as means±SEM. (C): Tumor growth of the syngeneic EMT6 breast cancer model is unresponsive to netrin-1 interference or Bcl-2 inhibition. EMT6 cells were engrafted in BALB/c mice and, once tumors reached a volume of 50 mm³, treated with isotypic (Iso-mAb) or NP137 antibodies and the Bcl-2 inhibitor Venetoclax, as single agents. Tumor volumes were measured at the indicated days after the first treatment (n=9, ^{ns}p>0.05, ordinary two-way ANOVA with Tukey's multiple comparisons test). Values are given as means±SEM. (D): Combined treatment is efficient to induce apoptosis in human ex vivo slices. RNA from breast tumor biopsies (n=8) was extracted and gene expression of netrin-1 (NTN1), UNC5B, Bcl-2 (BCL2) and Bcl-XL (BCL2L1) was assessed by quantitative RT-PCR. The heatmap represents basal mRNA levels for each patient, displayed as percentage of gene expression relative to the patient expressing the highest transcript levels. Patients in red responded to combined treatment with NP137 and Venetoclax, compared with single agent treatments.

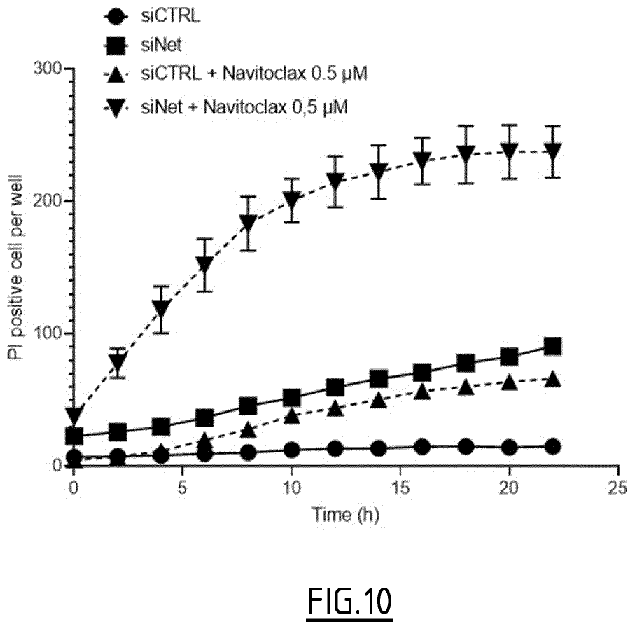

FIG. 10. Cell death assessment by combining netrin-1 silencing and Navitoclax. To assess cell death, Propidium iodide (PI) incorporation, normalized to cell confluence, was followed in real-time up to 24 hours by Incucyte Zoom technology for scramble (siCTRL), netrin-1 (siNet), scramble (siCTRL) and Navitoclax, netrin-1 (siNet) and Navitoclax.

### RESULTS

### Working Model for UNC5B-Mediated Mcl-1 Degradation and Activation of Intrinsic Apoptotic Pathway

Induction of UNC5B is able to activate, through COMMD2, the Cullin-RING-ligase (CRL) complex 3 (CRL3). Through unknown mechanisms involving NEDDylation of CRL3 and activation of the E2 ubiquitin-conjugating enzyme UBE2L6, UNC5B promotes ubiquitination and proteasomal degradation of Mcl-1, allowing Mitochondrial Outer Membrane Permeabilization (MOMP), release of cytochrome C, formation of apoptosome and finally activation of caspase-9, thus leading to apoptosis.

### COMMD2 is Required for UNC5B-Induced Cell Death and Tumor Suppression

To identify the pro-apoptotic pathways triggered by UNC5B, we generated stable HCT116 and HeLa cancer cell lines, inducible for UNC5B, using sleeping beauty-based vectors. Forced UNC5B expression upon doxycycline treatment, confirmed by western blot (FIGS. 2A and 2B), strongly induced apoptotic cell death in both models, as revealed by PI staining (FIGS. 1A and 2C), DNA fragmentation (FIGS. 1B and 2D) and caspase-3 activity (FIGS. 1C and 2E), compared to cells stably transfected with empty vector. This intense sensitivity to UNC5B induction indicates the presence of functional pro-apoptotic UNC5B signaling in HCT116 and HeLa cells. To confirm that UNC5B-induced apoptosis was not merely due to protein over-expression, we stably expressed the ligand netrin-1 in cells inducible for UNC5B. The constitutive expression of netrin-1 was sufficient to inhibit apoptosis that was otherwise observed upon UNC5B induction (FIG. 1B). Moreover, caspase inhibition by the pan-caspase inhibitor Q-VD-OPh completely prevented apoptosis induction by UNC5B (FIG. 2F).

We decided to exploit a stable HCT116 cell line, inducible for UNC5B, to perform a CRISPR-Cas9-based screening, allowing the identification of UNC5B pro-apoptotic effectors. For this purpose, we transduced sleeping beauty-modified HCT116 cells with a genome-wide lentiviral library (GeCKO library, (Sanjana et al., 2014; Shalem et al., 2014)). Upon doxycycline treatment and UNC5B induction, exclusively cell clones knocked-out of a gene required for UNC5B pro-apoptotic signaling should survive. Using Next Generation Sequencing (NGS), we identified inactivated genes in cell clones. Considering all genes presenting at least three small guide RNAs (sgRNAs) enriched compared to control cells and identified in more than 100 reads, we identified 75 genes potentially involved in UNC5B-induced apoptosis.

Following a literature-based selection we highlighted eight genes, listed in FIG. 2G. To confirm whether COMMD2 is required to UNC5B-induced cell death, we transfected stable HeLa and HCT116 cells with two distinct siRNA targeting COMMD2. Reduction of COMMD2 gene expression was sufficient to decrease DNA fragmentation upon UNC5B challenge with doxycycline, compared to cells transfected with control siRNA (FIG. 3A). Moreover, COMMD2 silencing inhibited the severe increase of PI-positive Hela cells, triggered by UNC5B over-expression, as well as caspase-3 activation observed in control cells (not shown). There is thus a specific role of COMMD2 in UNC5B pro-apoptotic pathways.

Next, we assessed the requirement of COMMD2 and UNC5B on tumor suppression associated with netrin-1 inhibition. For this purpose, we investigated the effect of NP137, a neutralizing anti-netrin-1 antibody able to induce cancer cell death and tumor growth inhibition in several preclinical models (Boussouar et al., 2020; Grandin et al., 2016; Sun et al., 2021), on stable knocked-out H322 cells engrafted in nude mice (FIG. 3B). Treatment of mice with the NP137 antibody strongly inhibited tumor growth in control cells, however tumors knocked-out of COMMD2 or UNC5B were less sensitive to anti-netrin-1 antibody, as shown by both tumor growth analysis and the percentage of tumor growth inhibition (FIGS. 3C, 3D). We confirmed that the NP137 anti-tumoral activity was associated with apoptosis induction, as demonstrated by immunohistochemical staining of cleaved, active caspase-3 (FIG. 3E). In contrast, in tumors knocked-out for COMMD2 or UNC5B, we failed to detect a similar increase in the percentage of caspase-3 positive cells upon treatment with NP137 antibody (FIG. 3E), further supporting the view that interfering with netrin-1/UNC5B interaction with NP137 triggers COMMD2-dependent cancer cell death associated with tumor growth inhibition.

### UNC5B Triggers Proteasomal Degradation of Mcl-1

Considering that we demonstrated that UNC5B-induced cell death requires COMMD2, we hypothesized that UNC5B could lead to ubiquitination and consequent proteasomal degradation of unknown target proteins. Along this line, treatment of stable Hela cells with proteasome inhibitors MG132 and bortezomib suppressed DNA fragmentation and caspase-3 activation triggered by UNC5B induction (FIGS. 4A and 4B). Searching for UNC5B target proteins degraded during apoptosis induction, we focused on the Bcl-2 family members, anti-apoptotic proteins whose cellular abundance is regulated via the ubiquitin-proteasome system (Kale et al., 2018; Singh et al., 2019). Western blot analysis revealed a specific decrease of Mcl-1 protein upon UNC5B induction in stable Hela cells, while we observed no such decrease on Bcl-2 and Bcl-XL protein levels (FIG. 4C). Moreover, treatment with the proteasome inhibitor bortezomib led to increased but similar Mcl-1 protein levels in both untreated and doxycycline-treated cells, supporting the view that the decrease of level of Mcl-1 associated with UNC5B induction is dependent on proteasome activity and thus is related to protein degradation. Interestingly, the inhibition of caspase activity, by means of Q-VD-OPh, was not sufficient to prevent Mcl-1 degradation triggered by UNC5B (FIG. 4C), even though it efficiently blocked UNC5B-induced apoptosis (see FIG. 2F), indicating that caspase activation is downstream of Mcl-1 degradation. In addition, inactivation of the COMMD2/CRL3 complex, by silencing COMMD2 or RBX1 gene expression, constrained Mcl-1 degradation in doxycycline-treated Hela cells (FIG. 4D).

To more formally demonstrate that degradation of Mcl-1 is specifically induced by UNC5B, we generated stable Hela cells inducible for UNC5B and constitutively expressing high levels of Mcl-1. Of interest, Mcl-1 over-expression was not sufficient to inhibit UNC5B-induced apoptotic cell death (FIG. 4E) and western blot analysis revealed that even ectopically over-expressed Mcl-1 is substantially degraded upon UNC5B induction, whereas in the same setting, overexpressed Bcl-2 and Bcl-XL proteins were not (FIG. 4F). Moreover, treatment with bortezomib restrained the degradation of ectopically-expressed Mcl-1 (FIG. 4G). To address the issue whether UNC5B may affect Mcl-1 degradation, we performed a cycloheximide (CHX) chase experiment. Time-dependent treatment of stable, Mcl-1-overexpressing, UNC5B-inducible Hela cells with CHX revealed an increased rate of Mcl-1 degradation in presence of UNC5B, resulting in a decreased half-life of Mcl-1 (108.5 min in control cells versus 37.2 min upon UNC5B induction, one phase decay equation) (FIG. 4H). Taken together, these results suggest that UNC5B drives proteasomal degradation of Mcl-1 through ubiquitin-ligase activity of the COMMD2/CRL3 complex.

### UNC5B-Mediated Mcl-1 Degradation Induces MOMP and Caspase-9 Activation

Mcl-1, as well as other Bcl-2 family members, acts as an anti-apoptotic protein by inhibiting the pro-apoptotic proteins BCL2 associated X, apoptosis regulator (BAX) and BCL2 antagonist/killer 1 (BAK1) and the consequent mitochondrial outer membrane permeabilization (MOMP) (Galluzzi et al., 2018). Following the cytoplasmic release of apoptotic factors residing in the mitochondrial intermembrane space, a pro-apoptotic complex, called the apoptosome, is formed, leading to caspase-9 activation and consequent execution of the intrinsic apoptotic program. As we have shown that UNC5B triggers Mcl-1 degradation, we hypothesized that apoptosis induced by UNC5B requires MOMP and caspase-9 activation. Indeed, UNC5B over-expression induced MOMP in stable Hela cells (FIG. 5A). Interestingly, silencing of COMMD2 and RBX1 inhibited MOMP triggered by UNC5B (FIG. 5A), suggesting that ubiquitin-ligase activity of the COMMD2/CRL3 complex is likely to be involved in UNC5B-triggered MOMP induction.

Next, we determined whether caspase-9, activated upon MOMP induction, is a mediator of UNC5B-triggered apoptosis. Stable Hela cells inducible for UNC5B were transfected with siRNAs targeting caspase-9, and upon treatment with doxycycline we evaluated cell death rate. Silencing of caspase-9 rescued the strong increase of PI-positive cells upon UNC5B induction (FIGS. 5B and 6A), as well as DNA fragmentation and caspase-3 activation (FIGS. 5C and 5D). In contrast, transfection of siRNAs targeting caspase-8 (the effector caspase involved in the extrinsic apoptotic pathway) failed to affect UNC5B-induced cell death (FIGS. 6B and 6C).

Dependence of UNC5B-induced death on caspase-9 suggests that UNC5B-induced MOMP triggers apoptosis through caspase-9 activation. Indeed, we showed that UNC5B triggered caspase-9, and not caspase-8, activation in stable Hela cells, as shown by caspase activity, performed using specific fluorescent substrates (FIG. 5E). Because we demonstrated that Mcl-1 degradation requires the activation of COMMD2/CRL3 complex by UNC5B, we determined whether UNC5B-dependent caspase-9 activation was a consequence of this event. Stable Hela cells were transfected with two distinct siRNAs targeting COMMD2, CUL3 and RBX1. Silencing the expression of COMMD2/CRL3 member complex completely suppressed caspase-9 activation observed upon UNC5B induction in scrambled-transfected cells (FIG. 5F). Moreover, treatment of stable HeLa cells with NEDD8 (Pevonedistat) or proteasome inhibitors (MG132 and bortezomib) was sufficient to prevent UNC5B-induced caspase-9 activation (FIG. 5G).

To confirm that Mcl-1 degradation and MOMP triggered by UNC5B were not merely the effect of cellular perturbation consequent to UNC5B over-expression, we determined whether caspase-9 knock-down affected MOMP or Mcl-1 degradation associated with UNC5B induction. Caspase-9 silencing (FIG. 6A), even though suppressing UNC5B-triggered apoptosis (FIGS. 5B, 5C, and 5D), was not sufficient to restrain mitochondria depolarization induced by UNC5B (FIG. 5H). As a control caspase 8 silencing (FIG. 6B) fails to inhibit cell death induced by UNC5B (FIG. 6C). Moreover, transfection of siRNA targeting caspase-9 in stable Hela cells did not prevent Mcl-1 degradation consequent to UNC5B induction (FIG. 5I). In a similar way, silencing of BAX and BAK1 (FIG. 6D), molecular mediators driving apoptosis through Mcl-1 inhibition to MOMP and caspase-9 activation, decreased UNC5B-dependent apoptosis induction (FIGS. 5J and 8D).

### Netrin-1 Interference Triggers Mcl-1 Degradation

To confirm that Mcl-1 degradation was associated to endogenously expressed disengaged UNC5B, we hypothesized that, in netrin-1 and UNC5B-expressing cancer cells, netrin-1 silencing should be associated with Mcl-1 degradation. Indeed, transfection of breast cancer MCF7 cells with an siRNA targeting netrin-1 induced Mcl-1 degradation (FIGS. 7A and 7B).

To move to a more relevant therapeutic setting, we assessed Mcl-1 degradation in vivo, using the EMT6 syngeneic breast carcinoma model. Indeed, EMT6 cells express endogenously netrin-1 and UNC5B. Stable EMT6 cells, constitutively over-expressing human Mcl-1, were engrafted in BALB/c mice and tumors bearing animals were treated i.v. with the therapeutic anti-netrin-1 antibody (NP137). Mcl-1 was measured by immunoblot from mice tumors and, of interest, netrin-1 blockade significantly reduced Mcl-1 protein levels, compared to control mice (FIGS. 7C and 7D).

The NP137 antibody is currently being assessed in several phase I and II clinical trials, in patients with advanced solid tumours. Paired biopsies from twelve patients treated with NP137 in the phase I trial were collected before the first infusion of NP137 (C1D1) and after two cycles of NP137 (1 month, C3D1). Mcl-1 presence was thus analysed by immunohistochemistry. NP137 administration reduced Mcl-1 levels in nine out of the twelve patient tumours tested (FIGS. 7E and F). Moreover, when comparing clinical benefit for the twelve patients from whom Mcl-1 detection was monitored (who experienced either a disease stabilization (SD: stable disease) or a disease progression (PD: progression disease) at the 6-weeks RESIST analysis), the six patients with SD showed Mcl-1 decrease upon NP137 treatment (FIGS. 7E and F), while we observed a decrease of Mcl-1 in only three of the six patients with PD. Together these data demonstrate that, both in animal model and in patients, netrin-1 blockade is triggering a tumoral specific Mcl-1 loss.

Combining netrin-1 interference and BH3 mimetics (Venetoclax and Navitoclax) inhibits tumor growth in preclinical in vivo and ex vivo models

BH3 mimetics represent a new class of therapeutic molecules, targeting Bcl-2 family proteins (Cerella et al., 2020). The Bcl-2 inhibitor ABT-199 (also known as Venetoclax) is currently the only BH3 mimetic approved by the United States Food and Drug Administration (FDA) and by the European Medicines Agency (EMA) for the treatment of chronic lymphocytic leukemia (CLL), small lymphocytic lymphoma (SLL), or acute myeloid leukemia (AML) (Souers et al., 2013). Several BH3 mimetics targeting Mcl-1 have been developed. Even though there was initial enthusiasm around Mcl-1 targeting, clinical trials on early Mcl-1 inhibitors have been recently suspended due to possible cardiac toxicity (Wei et al., 2020). BH3 mimetics used in monotherapy in solid tumors are less effective, possibly because solid tumors are less “primed” to apoptosis than hematopoietic tumors (Sarosiek et al., 2017) or because of compensation (van Delft et al., 2006). This is why the current strategy in solid tumors appears to be the combination of selective BH3 mimetics, in particular the combined treatment with Mcl-1 and Bcl-XL or Bcl-2 inhibitors (Soderquist et al., 2018). As we showed above that UNC5B receptor triggers Mcl-1 degradation in cancer cells, we hypothesized that triggering Mcl-1 degradation by UNC5B could potentiate the efficacy of Bcl-2 inhibitors. To confirm this hypothesis, first we generated stable breast cancer T47D cells, inducible for UNC5B, and we evaluated Venetoclax efficiency upon UNC5B induction. Indeed, UNC5B induction was sufficient to significantly reduce Venetoclax EC50 (FIG. 9A).

As we demonstrated that in MCF7 cells, expressing both netrin-1 and UNC5B, netrin-1 silencing was associated with Mcl-1 degradation, we hypothesized that, in these cells, interference with netrin-1 expression may bear to a potentiation of Bcl-2 inhibitor efficacy. Indeed, in contrast to netrin-1 interference or Bcl-2 inhibition alone, combining netrin-1 silencing and Venetoclax treatment triggered mitochondria depolarization, suggesting a potentiation of cell death induced by the inhibition of Bcl-2 (FIG. 9B).

Subsequently, to assess in vivo the anti-tumoral effect of the combined treatment in a conventional preclinical model, we exploited the EMT6 syngeneic breast carcinoma model. Indeed, these cells express high levels of both netrin-1 and UNC5B. However, interfering with netrin-1 through the anti-netrin-1 NP137 antibody has no effect on tumor growth in this model (not shown). EMT6 cells were engrafted in BALB/c mice and animal with tumors were treated with NP137 and Venetoclax. While the treatment with both the drugs in monotherapy had no effect on tumor growth, combined treatment markedly reduced tumor growth as compared to isotypic control antibody treatment (FIGS. 8A, 8B and 9C). This reduced tumor growth was associated with overall survival of the mice treated with the combination (FIG. 8C).

Finally, to confirm in tissue from human patients the efficiency of combined treatment, we evaluated the effect of netrin-1 interference and Bcl-2 inhibition in the ex vivo breast cancer organotypic culture technique, allowing the study of tumors kept in their natural microenvironment (de Graaf et al., 2010). Fresh tumor slices were treated with NP137 antibody and Venetoclax as single agents or combined, and apoptosis was quantified by immunohistochemistry analysis of cleaved Caspase-3. Treatment with NP137 antibody and Venetoclax significantly increased the percentage of cleaved caspase-3-positive cells, compared with netrin-1 interference or Bcl-2 inhibition alone (FIG. 8D).

Notably, combined treatment increased apoptosis rate in five out of eight tumor biopsies (FIGS. 8E and 8F). Remarkably, among the three patients not responding to combined treatment patient B21.015 expressed low levels of both netrin-1 and UNC5B, compared to the other patients, thus contributing to NP137 antibody resistance. Moreover, even though patient B21.030 presented high transcriptional levels of netrin-1 and UNC5B, resistance to combined treatment may be explained by relatively low levels of Bcl-2 (i.e., poor sensitivity to Venetoclax) and high expression of Bcl-XL, indicating resistance to Mcl-1 inhibition (Soderquist et al., 2018) (FIG. 9D). These results confirmed that Bcl-2 inhibition alone was not sufficient to trigger apoptosis in solid tumors (i.e., breast cancer human patients), requiring simultaneous interference with the netrin-1/UNC5B interaction, through NP137 antibody, to induce Mcl-1 degradation.

In addition, breast cancer HCC70 were transfected with scramble (siCTRL) or netrin-1 (siNet) targeting siRNAs. 24 hours after transfection, cells were detached, plated again in 24-well plate and treated with 0.5 UM of the Bcl-2/Bcl-XL/Bcl-w inhibitor Navitoclax (Tse et al, Cancer Res, 2008; Shoemaker et al, Clin Cancer Res, 2008). To assess cell death, propidium iodide (PI) incorporation, normalized to cell confluence, was followed in real-time up to 24 hours by Incucyte Zoom technology. Although netrin-1 silencing or Bcl-2/Bcl-XL/Bcl-w inhibition alone had a slight effect on cell death induction, combined treatment quickly and strongly triggered apoptosis (FIG. 10).

### Expression of UNC5 Receptors in Selected Cell Lines:

We have demonstrated that a lowering of netrin-1 presence in some tumoral cell lines such as MCF7 and H322 correlates with Mcl-1 degradation. We have also demonstrated a combined effect of netrin-1 depletion and BH3 mimetics in the tumoral cell line HCC70 on the cell death induction. We then have shown that UNC5B is well expressed in these cell lines as well as another netrin-1 receptor such as UNC5A and UNC5C.

The mRNA expression of the receptors UNC5A-C was evaluated by means of the Cancer Cell Line Encyclopedia (CCLE) database. Transcript abundance of the receptors, expressed as Reads Per Kilobase Million (RPKM), was assessed in breast (MCF7 and HCC70) and lung (H322) cancer cells.

TABLE
Gene expression (RPKM)

| | MCF7 | HCC70 | H322 |
|---|---|---|---|
| UNC5A | 0.854 | 0.000 | 0.157 |
| UNC5B | 0.540 | 1.297 | 0.575 |
| UNC5C | 0.095 | 1.917 | 0.305 |

Except UNC5B, whose expression is high and stable in all the three cell lines analyzed, at least one receptor between UNC5A and UNC5C is also expressed. It is thus deemed other UNC5 receptors, especially UNC5A and UNC5C are plausibly involved in cell death induced by netrin-1 interfering or silencing, alone or in combination with BH3 mimetics.

### Discussion

The dependence receptor UNC5B induces apoptosis conditionally in cancer cells. Using a CRISPR-Cas9 screening, we identified the protein COMMD2 as a pro-apoptotic mediator of UNC5B-induced cell death. In the absence of netrin-1, UNC5B triggers COMMD2 activation by still unknown mechanisms. Once activated, COMMD2 interacts with the CRL3 complex which then ubiquitinates Mcl-1 and causes its degradation by the proteasome. This results in a shift of the pro-apoptotic balance, leading to mitochondrial permeabilization and successive activations of caspase-9 and caspase-3, and finally to apoptosis of the cancer cell. To date, several ubiquitin ligases are known to degrade Mcl-1, including Mule, SCFR-TrCP, SCFFBW7, TRIM17, APC/CCdc20, MARCH5 and Parkin (Mojsa et al., 2014; Senichkin et al., 2020). However, the CRL3 complex has not previously been described in this context. Here, for the first time, we demonstrate the involvement of this E3 ligase in the degradation of Mcl-1.

The E3 ligase family of CRLs is mainly regulated by a neddylation cycle, allowing their activation and inactivation (Duda et al., 2011). CRL E3 ligases are activated by covalent binding of a NEDD8 protein (Enchev et al., 2015). Subsequently NEDD8 can be dissociated from the complex by the action of the COP9 (constitutive photomorphogenesis 9) signalosome, which allows the CAND1 protein to interact with the non-neddylated complex and inhibit its activity (Duda et al., 2011). Interestingly COMMD1 inhibits the binding of CAND1 by associating with Cullin-2, and consequently activates the CRL2 complex (Mao et al., 2011). In addition, COMMD2 specifically interacts with Cullin-3, but not Cullin-2 (Mao et al., 2011). Our data demonstrate that COMMD2, as well as the members of CRL3 complex, are required for cell death induced by UNC5B, whereas COMMD1 and other CRLs did not participate in the UNC5B pro-apoptotic pathway. Furthermore, we were able to demonstrate that inhibition of neddylation, via treatment with pevonedistat, a specific inhibitor of NEDD8, prevented UNC5B-induced cell death. Since CAND1 binds and inhibits virtually all Cullins (Duda et al., 2011), we can thus hypothesize that COMMD2, by interacting specifically with CRL3, inhibits the interaction of CAND1 with Cullin-3, and thus allows its activation by neddylation. However, how UNC5B favorizes COMMD2 interaction with CRL and activates these complexes remains unclear.

From a therapeutic point of view, our results suggest that interfering with the binding between netrin-1 and UNC5B, using the neutralizing anti-netrin-1 antibody NP137, constitutes an original means of degrading Mcl-1, thus providing an interesting alternative to the BH3-mimetics targeting Mcl-1, currently being developed. By degrading Mcl-1 in an indirect but tumor-specific way, NP137 antibody is an attractive alternative to BH3-mimetic compounds. Indeed, using NP137, Mcl-1 degradation is deemed occurring only in netrin-1 expressing cells, and the preclinical development of NP137 has revealed an absence of tissue cross-reactivity in heathy adult individuals. Moreover, the safety of this antibody has already been tested in clinical phase I, and has shown an extremely good safety profile (Cassier et al., 2019). NP137, by preferentially targeting tumor cells overexpressing netrin-1, has a considerable advantage over chemical inhibitors, which indiscriminately target both tumor and healthy cells. The good tolerance to the antibody in patients offers the possibility of using it in combination with other molecules.

Another interesting approach is to target other Bcl-2 family members in addition to Mcl-1. While the vast majority of solid tumors are resistant to the use of a single inhibitor targeting Bcl-2, Bcl-XL, or Mcl-1, the combination of inhibition of two or even all three proteins is highly effective (Soderquist et al., 2018). The combination of Mcl-1 and Bcl-XL inhibitors, in particular, appears to be effective in a large portion of tumors and thus offers promise. However, this combination has been reported to be lethal in mouse models, due to acute liver toxicity (Weeden et al., 2018). Using the antibody against netrin-1 represents a more selective BH3 mimetic targeting Mcl-1, and therefore decreases the toxicity of the co-treatment and allows combinations with other BH3 mimetics, as we demonstrated here in several preclinical and ex vivo tumor models treated with NP137 and the Bcl-2 inhibitor Venetoclax.

Another BH3 mimetic has been assayed. Navitoclax (ABT-263) is known to induce inhibition of Bcl-2/Bcl-XL/Bcl-w. The combination of netrin-1 silencing and Navitoclax quickly and strongly triggered apoptosis (FIG. 9).

### Methods:

### Cell Lines, Transfection Procedures, Reagents

Human colorectal HCT116 and cervical HeLa cancer cell lines were cultured in DMEM medium (Life Technologies, Carlsbad, CA, USA) containing 10% fetal bovine serum. HEK 293T cells were cultured in DMEM medium (Life Technologies) containing 10% fetal bovine serum, non-essential amino acids (NEAA, Life Technologies) and 50 μg/ml Geneticin (Life Technologies). Human lung H322 cancer cells were cultured in RPMI 1640+Glutamax medium (Life Technologies) containing 10% fetal bovine serum. For RNA interference experiments, cell lines were transfected using lipofectamine RNAiMAX reagent (Life Technologies). For plasmid transfection, HCT116 cell line was transfected using lipofectamine Plus reagent (Life Technologies); HeLa cell line was transfected using jet prime (PolyPlus-Transfection). All transfections were performed according to the manufacturer's protocol. BH3 mimetic Venetoclax (ABT-199) and NEDD8 inhibitor pevonedistat were purchased from Selleck Chemicals GmbH (Munich, Germany). Proteasome inhibitors MG132 and Bortezomib were purchased from Sigma-Aldrich (St. Louis, MO, USA). Caspase inhibitor QVD was obtained from Adooq Bioscience (Irvine, CA, USA).

### Generation of Stable Cell Lines

For the generation of stable inducible cell lines, histidine-tagged rat UNC5B coding sequence was cloned at Sfil sites in the pSBtet-GH plasmid, a sleeping beauty-based vector allowing the doxycycline inducible expression of UNC5B, as well as the constitutive expression of rtTA protein, hygromycin resistance gene and green fluorescent protein (GFP), expressed under the control of the synthetic RPBSA promoter. HCT116, HeLa, and T47D cell lines were co-transfected with UNC5B-pSBtet-GH and a sleeping beauty transposase expression vector (SB100X). Sleeping beauty transposase expression vector was a generous gift of Prof. Rolf Marschalek (Goethe-University of Frankfurt, Germany). pSBtet-GH was a gift from Eric Kowarz (Addgene, Watertown, MA, plasmid #60498; http://n2t.net/addgene: 60498; RRID: Addgene_60498). Stable cell clones were selected for 7 days in hygromycin-containing medium. Then stable UNC5B cell lines were sorted by flow cytometry to select high GFP-positive cells. All the stable inducible cell lines were further tested for UNC5B expression in response to doxycycline.

Stable cell line constitutively expressing netrin-1 were generated by lentiviral infection, using self-inactivating HIV-1-derived vector (pWPXLd), encoding netrin-1 under the control of Human Elongation Factor-1 alpha (EF-1 Alpha) promoter. For the generation of HCT116 stable cell line inducible for UNC5B used for the screening, a pCW57.1 vector was used, and stable cell line was generated by lentiviral infection. This lentiviral vector allows the doxycycline inducible expression of UNC5B and the constitutive expression of rtTA protein and hygromycin resistance gene. pCW57.1 (Addgene, plasmid #41393; http://n2t.net/addgene: 41393; RRID: Addgene_41393).

For the generation of stable H322 cell lines knocked-out for UNC5B or COMMD2, different small guide RNAs (sgRNA) were designed using the sgRNA designer tool CRISPick (CRISPRi), available at (https://) portals.broadinstitute.org/gppx/crispick/public site. sgRNAs were designed close to UNC5B or COMMD2 promoters. BsmBI linker were added to sgRNAs and the oligonucleotides were then annealed by incubation for 3 min at 90° C. and 15 min at 37° C. and inserted in BsmBI-digested pLV hU6-sgRNA hUbC-dCas9-KRAB-T2a-Puro vector (Thakore et al., 2015), allowing expression of defective Cas9 enzyme (dCas9) fused to the Krüppel-associated box (KRAB) transcriptional repressor (dCas9-KRAB). pLV hU6-sgRNA hUbC-dCas9-KRAB-T2a-Puro (Addgene, plasmid #71236; (http://) n2t.net/addgene: 71236; RRID: Addgene_71236).

### Quantitative RT-PCR

Total RNAs were extracted using NucleoSpin® RNA Plus Kit (Macherey Nagel, Düren, Germany) according to manufacturer's protocol. RT-PCR reactions were performed with PrimeScript RT Reagent Kit (Takara Bio Europe, Saint-Germain-en-Laye, France). 500 ng total RNA was reverse-transcribed using the following program: 37° C. for 15 min and 85° C. for 5 sec. For expression studies, the target transcripts were amplified in LightCycler®2.0 apparatus (Roche Applied Science, Penzberg, Germany), using the Premix Ex Taq (probe qPCR) Kit (Takara Bio Europe), according to manufacturer instructions. Expression of target genes was normalized to TATA binding protein (TBP) and beta-glucuronidase (GUSB) genes, used as housekeeping genes. The amount of target transcripts, normalized to the housekeeping gene, was calculated using the comparative CT method. A validation experiment was performed, in order to demonstrate that efficiencies of target and housekeeping genes were approximately equal. Sequences of the primers are available upon request.

### Western Blotting Analysis

For immunoblotting analysis, cells were lysed by sonication in SDS buffer (10 mM Tris-HCl pH 7.4, 10% glycerol, 5% SDS, 1% TX-100, 100 mM DTT) in the presence of protease inhibitor cocktail (Roche Applied Science). Protein extracts (20-50 μg per lane) were loaded onto 4-15% SDS-polyacrylamide gels (Bio-rad, Hercules, CA, USA) and blotted onto nitrocellulose sheets using Trans-Blot Turbo Transfer System (Bio-rad). Nitrocellulose sheets were blocked with 10% non-fat dried milk in PBS/0.1% Tween 20 (PBS-T) for 1 hour and then incubated over-night at 4° C. with the following antibody: rabbit monoclonal antibody a-UNC5B (1:1000, clone D9M7Z, #13851, Cell Signaling, Danvers, MA); mouse monoclonal antibody a-B-actin, HRP-conjugated (1:10000, clone BA3R, #MA5-15739-HRP, Life Technologies); rabbit monoclonal antibody a-Mcl-1 (1:1000, clone D5V5L, #39224, Cell Signaling); rabbit monoclonal antibody a-Bcl-XL (1:1000, clone 54H6, #2764, Cell Signaling); rabbit polyclonal antibody a-Bcl-2 (1:1000, clone 21, #783, Santa Cruz Biotechnology, Dallas, TX, USA); rabbit polyclonal antibody a-COMMD2 (1:1000, HPA044190, Sigma-Aldrich); rabbit monoclonal antibody a-Ku80 (1:1000, clone C48E7, #2180, Cell Signaling). After three washes with PBS-T, membranes were incubated with the appropriate HRP-conjugated secondary antibody (1:10000, Jackson ImmunoResearch, Suffolk, UK) for 1 h. Detection was performed using West Dura Chemiluminescence System (Life Technologies). Membranes were imaged on the ChemiDoc Touch Imaging System (Bio-rad). Membranes were imaged on the ChemiDoc Touch Imaging System (Bio-rad). Protein band quantification was performed with Image Lab 6.1 software (Bio-rad), considering the adjusted volume intensity normalized with housekeeping genes.

### Cell Death Assays

For propidium iodide (PI) staining, stable inducible cell lines were seeded in 48-well-plates at density of 25*10³ cells/well. The next day, cells were treated with doxycycline and 0.33 mg/ml PI in serum-free (HCT116) or 10% serum (HeLa) medium and the plates were transferred into Incucyte Zoom instrument (Essen Bioscience, Royston, UK). Images of living cells and PI-stained dead cells were taken every two or three hours and PI-stained cell numbers were calculated.

For DNA fragmentation analysis (SubG1), HCT116 and HeLa cell lines were plated onto 6-well-plates at a density of 2*10⁵ cells per well. The following day, cells were transfected with siRNA, ad described above, and incubated in serum-free medium for 24 hours. Transfected cells were then treated with doxycycline in 10% serum medium and further incubated for 18 hours. Then cells were collected, fixed in 70% ethanol and incubated at −20° C. for at least one night. Afterwards, cells were washed twice with PBS and incubated for 15 min at room temperature in PBS containing 2 mg/ml RNase A and 40 mg/ml PI. DNA content was evaluated by fluorescence activated cell sorting (FACS) analysis on a FACScalibur or FacsCantoll (BD Bioscience, Franklin Lakes, NJ, USA).

Caspase-3 and -9 activity assays were performed as described previously (Paradisi et al., 2013; Paradisi et al., 2009). Briefly, HCT116 and HeLa cells were seeded and treated as for DNA fragmentation assay. Cells were then collected and caspase-3/caspase 9 activity assays were performed using the Caspase-3/Caspase-9 Fluorometric Assay Kit (BioVision, Milpitas, CA, USA), according to the manufacturer's instructions. Caspase activity (activity/min/mg of protein) was calculated from a 1 h kinetic cycle reading on a spectrofluorometer (405 nm/510 nm, Infinite F500, Tecan, Männedorf, Switzerland).

### Mitochondrial Potential Assay

HCT116 and Hela cells were seeded and treated as for DNA fragmentation assay. Cells were then collected and washed with PBS. 1×10⁶ cells for each sample were suspended in 1 mL PBS containing JC-1 dye (2.5 g/mL, Chemometec, Allerod, Denmark). After incubation at 37° C. for 15 minutes, stained cells were centrifuged at 800×g for 5 min at room temperature and supernatant was removed completely without disturbing the cell pellet. Afterwards, cell pellet was resuspended in 1 ml PBS for washing, then centrifuged at 800×g for 5 min at room temperature and supernatant was removed again. Washing step was repeated twice. Later, cell pellet was resuspended by pipetting in 250 μl PBS buffer containing 1 μg/ml DAPI. The final stained cells were analyzed by using NucleoCounter NC-3000 (Chemometec).

### Conditional Medium Production

HCT116 stable cells constitutively expressing netrin-1 were plated in 100 mm-dishes (3*10⁶ cells per dish). 24 hours later, cell medium was collected and centrifuged at 1200×g for 5 minutes and supernatant was recovered as netrin-1 conditional medium.

### CRISPR-Cas9 Screening

### Gecko Library

Human GeCKOv2 CRISPR knockout pooled library (targeting 19.050 genes and divided two half-libraries, each containing 3 sgRNAs against each gene) was a gift from Feng Zhang (Addgene).

### DNA Amplification of the Library

Each lentiviral library (A and B) was amplified and prepared separately following the same protocol. 2 μL of 50 ng/μL of each GeCKO library were electroporated in Endura electrocompetent cells (Lucigen, Middleton, WI, USA). Electroporation in competent cells was made four times for each library. Cells were resuspended in recovery media and placed in shaking incubator for 1 hour at 37° C. For each library, electroporated cells were plated on two pre-warmed 600 cm² Luria broth (LB) agar plates with ampicilin and were grown for 16 hours at 37° C. In parallel, a 30.000-fold dilution of the full transformation was plated to estimate transformation efficiency and ensure that full library representation is preserved. The total number of colonies was at least 3.106 corresponding approximatively to 50 colonies per construct in each GeCKO library. Colonies were harvested by adding 10 mL of LB medium onto each plate and scrapped with a cell spreader. Liquid with scrapped colonies were collected into a tube and the procedure was repeated a second time on the same plate with additional 10 ml of media. Appropriated number of plasmid preparation (NucleoBond Xtra, Macherey Nagel) were performed and the concentration of each library was determined by UV-VIS spectroscopy (NanoDrop 2000, Thermo Fischer Scientific, Waltham, MA, USA). Plasmid libraries A and B were pooled in equimolar proportions for lentivirus production.

### Lentivirus Production and Titration

HEK 293T cells were seeded at 5.10⁶ cells per 150 mm-dish one day prior to transfection. Cells were transfected using Xtreme gene reagent (Roche Applied Science) with three expression vectors, respectively, 10 μg of phCMV-VSV-g, 15 μg phCMV-Gag-Pol-HIV, 20 μg GeCKO plasmid library. Six hours after transfection, medium was replaced. Two viral harvests were collected at 40 hours and 60 hours after transfection. The two harvests were pooled, centrifuged and filtered through a 0.45 μm filter. Aliquots from the cleared supernatant were stocked at −80° C. Lentivirus production was titrated by infecting HCT116 cells in 12-well plates with several different volumes of lentivirus. The day after infection, each well was split into two wells: one receiving puromycin at 0.8 μg/mL, the other one no puromycin. 72 hours after selection, cells were counted to calculate the percent of transduction and determine the viral titer.

### Cell Library Generation

The day before transduction, HCT116 cells were plated in eight 12-well plates at a concentration of 10⁵ cells per well. Cells were then transduced with a volume of GeCKO lentivirus library determined earlier for achieving a multiplicity of infection (MOI) between 0.3 and 0.5. HCT116 cells were transduced by spin infection during 1 hour and 30 minutes at 37° C. in complete DMEM medium supplemented with 8 μg polybrene. The following day, cells were trypsinized, seeded into eight 150 mm-dishes and treated with puromycin at 0.8 μg/mL. As a control of selection, non-transduced cells were plated and treated with puromycin at 0.8 μg/mL.

### UNC5B Challenge in Library-Transduced Cells

After puromycin selection, a part of the cell library was lysed for genomic extraction and sequencing. Two conditions of 24.106 cells each were plated in serum-free DMEM, and treated or not with 2 μg/ml of doxycycline. In parallel, a control condition of stable HCT116 cell line, inducible for UNC5B and not transduced with CRISPR library, was induced with the same doxycycline concentration. When all cells were dead in the control condition, surviving cells of the two conditions of the library were amplified for three days and then lysed for genomic extraction and sequencing.

### Genomic Extraction and DNA Amplification

Genomic DNA (gDNA) was extracted from the cell library before UNC5B induction and from surviving cells after UNC5B induction using NucleoSpin Tissue Kit (Macherey-Nagel). The amplification of pooled sgRNA isolated from cell samples was carried out using Clone Amp HiFi enzyme (Takara Bio Europe). Primers were designed in order to amplify a 150 bp region surrounding sgRNA sequences and add a barcode sequence, specific for each sample, and an adapter needed to immobilize PCR product in the NGS flow cells. Three separate PCRs with 250 ng of gDNA each were realized for each sample. PCR products were purified with PCR Clean-up/Gel extraction kit (Macherey Nagel) then run on agarose gel following by a new purification step with the same kit. A last step of purification (AMPure, Beckman Coulter, Brea, CA, USA) was done prior sequencing.

### Next Generation Sequencing and Analysis

Next Generation Sequencing (NGS) was performed by the Sequencing platform of Institute of functional genomics of Lyon (IGFL, Lyon) using Ion PI HiQ Chef Kit and Ion PI HiQ chip v3 (100pb-80M reads) on Ion Torrent platform (Thermo Fischer Scientific). The amplified samples were pooled together on the sequencing chip for the run and differentiated with barcode identification. Raw data from sequencing were processed to eliminate incorrect sequences and trim sequences flanking the sequence of the sgRNA. The trimmed sequences were blasted against the GeCKO library database with determined parameters (95% identity on 90% of the length) and only the first hit was kept. Finally, a list was obtained with the number of reads for each sgRNA for each experimental condition. For a given condition, the number of reads of each sgRNA was normalized by the total number of reads in this condition. Reads were then normalized by subtracting the number of reads in UNC5B-induced condition to the control condition. Finally, sgRNA sequences with more than 100 normalized reads were selected, and then genes with more than 3 sgRNA sequences were selected and confirmed.

### Xenografts in Nude Mice

5-week-old female athymic NMRI nude mice were obtained from Janvier Labs (Saint Bertevin, France). The mice were maintained in a Specific and Opportunistic Pathogen-Free (SOPF) animal facility. Parental or stable H322 cells, deleted for COMMD2 or UNC5B were implanted by subcutaneous injection of 5*10⁶ cells suspended in 100 μl of PBS into the right flank of nude mice. When tumors reached 100 mm³, mice were intravenously injected three times per week with 10 mg/Kg of anti-netrin-1 (NP137) or isotypic control antibodies for 36 days. Tumor size was measured three times per week with a caliper. Tumor volume was calculated according to the formula v=0.5× (l×w2), where v is volume, l is length, and w is width. All experiments were performed in accordance with relevant guidelines and regulations of animal ethics committee (Authorization APAFIS no: 28723; accreditation of laboratory animal care by CECCAP, ENS Lyon-PBES, France).

### EMT6 Syngeneic Breast Tumor Model

Five-week-old Balb/c female were purchased from Janvier Labs. EMT6 cells were implanted by subcutaneous injection of 5*105 cells suspended in 100 μl of PBS. When tumors reached 50 mm³, mice were intravenously injected with a loading dose of 20 mg/Kg of NP137 or isotypic control antibodies for three consecutive days, followed by a maintenance dose of 10 mg/Kg three times per week for 12 days Venetoclax (5 mg/Kg, Selleck Chemicals) or DMSO (resuspended in 50% PEG 300+5% Tween 80 in distilled water, Selleck Chemicals GmbH) were administrated by oral gavage five times per week during two weeks. Tumor size was measured three times per week with a caliper. Tumor volume was calculated according to the formula v=0.5×(l×w2), where v is volume, l is length, and w is width. All experiments were performed in accordance with relevant guidelines and regulations of animal ethics committee (Authorization APAFIS n°: 28780; accreditation of laboratory animal care by CECCAP, ENS Lyon-PBES, France).

For in vivo Mcl-1 degradation, EMT6 cells were implanted by subcutaneous injection of 5*10⁵ cells suspended in 100 μl of PBS. When tumors reached 100 mm³, mice were intravenously injected with 20 mg/Kg of NP137 or isotypic control antibodies. Eight hours after injection, mice were sacrificed and tumors were collected and immediately frozen at −80° C.

### Ex Vivo Breast Cancer Organotypic Culture

A total of 8 fresh human mammary samples were obtained from chemotherapy-naïve patients with invasive carcinoma after surgical resection at the Centre Léon Bérard (CLB, Lyon, France). All of the patients signed informed consent letters, as required by the French Bioethics law, approving the use of their samples for research, and the investigation was approved by the ethic committee of CLB. After surgical resection and macroscopic anatomopathological examination, the tumor tissue was immediately kept in 4° C. in PBS and transported to the laboratory. Afterwards, the tumor was examined on a routine basis and classified by a board-certified pathologist. After removing excess of necrotic tissue, 200 mm³ fresh tumor fragment was included in agarose matrix. Automated slicing was performed using MICROM HM650V Vibratome (Thermo Fisher Scientific, Walldorf, Germany) with slice thickness set at 250 μm, vibration amplitude at 8 mm and slicing speed at 0.6 mm/sec. Slices were cultured within 2 hours after the tumor was removed from the patient. Culturing was performed at 5% CO2 at 37° C. and at atmospheric oxygen levels. Slices were incubated individually using 24-well plates for 24 hours in DMEM medium supplemented with 5% Fetal Bovine Serum, 1% GlutaMAX, 1% penicillin/streptomycin, 1% MEM, 1% HEPES (all from Life Technologies). Several successive slices of each fresh tumor were then treated for 24 hours with 10 μg/ml of anti-netrin-1 (NP137) or isotypic control (NP001) antibodies, and 1 μM of Venetoclax in DMSO. Apoptosis in treated slices was evaluated by immunohistochemical staining of active caspase-3.

### Immunohistochemical Staining

For histological examination of tumours engrafted in nude mice, ex vivo breast cancer slices, and from human clinical trial, tumours were fixed in 10% buffered formalin and embedded in paraffin. 4-μm-thick tissue sections of formalin-fixed, paraffin-embedded tissue were prepared according to conventional procedures. Sections were then stained with haematoxylin and eosin and examined with a light microscope. Immunohistochemistry was performed on an automated immunostainer (Ventana Discovery XT, Roche, Meylan, France) using Omnimap DAB Kit according to the manufacturer's instructions. Sections were incubated with a rabbit anti-cleaved caspase-3 (diluted at 1:500, #9661S, Cell Signaling) or rabbit anti-Mcl-1 (diluted 1:500, #HPA031125, Sigma-Aldrich) antibodies. HRP-conjugated anti-rabbit antibody was applied on sections. Staining was visualized with DAB solution with 3,3′-diaminobenzidine as a chromogenic substrate. Then, the sections were counterstained with Gill's haematoxylin. Finally, sections were scanned with panoramic scan II (3D Histech, Budapest, Hungary) at 20×. Quantification of caspase-3- and Mcl-1-positive cells was performed with HALO software and adapted algorithms cytonuclear v2.0.8 6 (Indica Labs, Albuquerque, NM, USA). MCL1 intensity, has been calculated in percentage over the surface of tumour cells. All immunohistochemical staining and analysis were performed by the Research Pathology Platform East (Plateforme Anatomopathologie Recherche, Université de Lyon, Université Claude Bernard Lyon 1, INSERM 1052, CNRS 5286, Centre Léon Bérard, Centre de recherche en cancérologie de Lyon (CRCL), Lyon, 69373, France).

### Statistical Analysis

Statistical tests are indicated in each figure and were performed using GraphPad Prism version 9.4.1 for Windows (GraphPad Software, La Jolla, CA, USA). Survival curves were produced according to the Kaplan-Meier method on the GraphPad software.

### REFERENCES

Adams, J. M., and Cory, S. (2018). Cell death and differentiation 25, 27-36.
Ashkenazi, A., et al. (2017). Nature reviews Drug discovery 16, 273-284.
Boussouar, A., et al. (2020). Cancer research 80, 747-756.
Bredesen, D. E., et al. (2005). Cell death and differentiation 12, 1031-1043.
Brennan, M. S., et al. (2018). Blood 132, 1573-1583.
Broutier, L., et al. (2016). EMBO molecular medicine 8, 96-104.
Cassier, P., et al. (2019). 4390. Annals of Oncology 30, v159.
Castets, M., et al. (2009). Developmental cell 16, 614-620.
Cerella, C., et al. (2020). Trends Pharmacol Sci 41, 793-814.
de Graaf, I. A., et al. (2010). Nature protocols 5, 1540-1551.
Delbridge, et al. (2016). Nature reviews Cancer 16, 99-109.
Delloye-Bourgeois, C., et al. (2009a). Journal of the National Cancer Institute 101, 237-247.
Delloye-Bourgeois, et al. (2009b). The Journal of experimental medicine 206, 833-847.
Duda, D. M., et al. (2011). Current opinion in structural biology 21, 257-264.
Enchev, R. I., et al. (2015). Nature reviews Molecular cell biology 16, 30-44.
Ewald, L., et al. (2019). Cell death & disease 10, 917.
Fitamant, J., et al. (2008). PNAS the United States of America 105, 4850-4855.
Galluzzi, L., et al. (2018). Molecular mechanisms of cell death: recommendations of the
Nomenclature Committee on Cell Death 2018. Cell death and differentiation.
Goldschneider, D., and Mehlen, P. (2010). Oncogene 29, 1865-1882.
Grandin, M., et al. (2016) . . . . Cancer cell 29, 173-185.
Guenebeaud, C., et al. (2010). Molecular cell 40, 863-876.
Hanahan, D., and Weinberg, R. A. (2000). The hallmarks of cancer. Cell 100, 57-70.
Juin, P., et al. (2013). Nature reviews Cancer 13, 455-465.
Kale, J., et al. (2018). Cell death and differentiation 25, 65-80.
Kotschy, A., et al. (2016). Nature 538, 477-482.
Lai Wing Sun, et al. (2011). Development 138, 2153-2169.
Leverson, J. D., et al. (2015). Science translational medicine 7, 279ra240.
Llambi, F., et al. (2001). The EMBO journal 20, 2715-2722.
Llambi, F., et al. (2005). The EMBO journal 24, 1192-1201.
Mao, X., et al. (2011). The Journal of biological chemistry 286, 32355-32365.
Mehlen, P., and Bredesen, D. E. (2004). The dependence receptor hypothesis. Apoptosis:
an international journal on programmed cell death 9, 37-49.
Mehlen, P., and Bredesen, D. E. (2011). Dependence receptors: from basic research to drug
development. Science signaling 4, mr2.
Mehlen, P., et al. (2011). Nature reviews Cancer 11, 188-197.
Mehlen, P., and Puisieux, A. (2006). Nature reviews Cancer 6, 449-458.
Mehlen, P., et al. (1998). Nature 395, 801-804.
Merino, D., et al. (2018). Cancer cell 34, 879-891.
Mérino, D., et al. (2012). Oncogene 31, 3392-3396.
Mojsa, B., et al. (2014). Cells 3, 418-437.
Negulescu, A. M., and Mehlen, P. (2018) The FEBS journal 285, 3909-3924.
Opferman, J. T., et al. (2005). Science 307, 1101-1104.
Paradisi, A., et al. (2013). EMBO molecular medicine 5, 1821-1834.
Paradisi, A., et al. (2008). Gastroenterology 135, 1248-1257.
Paradisi, A., et al. (2009). PNAS United States of America 106, 17146-17151.
Sanjana, N. E., et al. (2014). Nature methods 11, 783-784.
Sarosiek, K. A., et al. (2017). Cancer cell 31, 142-156.
Scheffold, A., et al. (2018). Venetoclax: Targeting BCL2 in Hematological Cancers. Recent results in cancer research 212, 215-242.
Senichkin, et al. (2020). Cell death and differentiation 27, 405-419.
Shalem, O., et al. (2014). Science 343, 84-87.
Singh, R., et al. (2019). Nature reviews Molecular cell biology 20, 175-193.
Soderquist al. (2018). Nature comm. 9, 3513, DOI: 10.1038/s41467-018-05815-z.
Souers, A. J. et al. (2013). Nature medicine 19, 202-208.
Sun, Y. et al. (2021). PNAS United States of America 118.
Sung, P. J. et al. (2019). Cancer research 79, 3651-3661.
Thakore, P. I. et al. (2015). Nature methods 12, 1143-1149.
Thiebault, K. et al. (2003). PNAS United States of America 100, 4173-4178.
van Delft, M. F. et al. (2006). Cancer cell 10, 389-399.
Wang, R. et al. (2009). Molecular cell 33, 692-703.
Weeden, C. E. et al. (2018). Oncogene 37, 4475-4488.
Wei, A. H. et al. (2020). Blood reviews 44, 100672.
Zhang, H. et al. (2007). Cell death and differentiation 14, 943-951.

1.-38. (canceled)

39. A pharmaceutical composition comprising an anti-netrin-1 antibody, or an antigen-binding fragment thereof, an anti-apoptotic protein of the Bcl-2 family, in particular a Bcl-2 inhibitor, and a pharmaceutically acceptable vehicle or excipient.

40. The composition of claim 39, wherein the anti-netrin-1 antibody, or antigen-binding fragment thereof:
(i) specifically binds to a polypeptide of amino acid sequence SEQ ID NO: 3 or 35,
(ii) or comprises
a variable domain VH comprising:
a H-CDR1 having a sequence set forth as SEQ ID NO: 5;
a H-CDR2 having a sequence set forth as SEQ ID NO: 6;
a H-CDR3 having a sequence set forth as SEQ ID NO: 7;
a variable domain VL comprising:
a L-CDR1 having a sequence set forth as SEQ ID NO: 8;
a L-CDR2 having a sequence YAS;
a L-CDR3 having a sequence set forth as SEQ ID NO: 9;
(iii) or comprises
a variable domain VH comprising:
a H-CDR1 having a sequence set forth as SEQ ID NO: 28;
a H-CDR2 having a sequence set forth as SEQ ID NO: 29;
a H-CDR3 having a sequence set forth as SEQ ID NO: 30;
a variable domain VL comprising:
a L-CDR1 having a sequence set forth as SEQ ID NO: 31;
a L-CDR2 having a sequence set forth as SEQ ID NO: 32;
a L-CDR3 having a sequence set forth as SEQ ID NO: 9.

41. The composition of claim 39, wherein the anti-netrin-1 antibody or antigen-binding fragment thereof is selected from the group consisting of anti-netrin-1 antibodies and antigen-binding fragments thereof comprising pairs of VH and VL amino acid sequences SEQ ID NO: 20 and 14, SEQ ID NO: 21 and 15, SEQ ID NO: 22 and 16, SEQ ID NO: 23 and 17, SEQ ID NO: 24 and 17, SEQ ID NO: 25 and 16, SEQ ID NO: 26 and 17, SEQ ID NO: 22 and 17, SEQ ID NO: 25 and 18, SEQ ID NO: 21 and 16, or SEQ ID NO: 27 and 19.

42. The composition of claim 39, wherein the anti-netrin-1 antibody comprises a Human IgG1 Constant heavy chain (CH) and/or a Human IgG1 Constant light chain (CL), in particular a human kappa constant domain.

43. The composition of claim 39, wherein the anti-netrin-1 antibody comprising a VH of sequence of SEQ ID NO: 22 and a VL of sequence SEQ ID NO: 16.

44. The composition of claim 39, further comprising a human IgG1 CH, and a human IgG1 CL Kappa.

45. The composition of claim 39, further comprising a human IgG1 CH Genbank AEL33691.1 modified R97K, and a human IgG1 CL Kappa Genbank CAC20459.1.

46. The composition of claim 39, wherein the inhibitor of an anti-apoptotic protein of the Bcl-2 family is Venetoclax or Navitoclax.

47. A method of treatment of a Mcl1+ and netrin-1 receptor+, especially UNC5B+, cancer comprising administering to a patient having such cancer an efficient amount of an anti-netrin-1 antibody, or an antigen-binding fragment thereof, and of an inhibitor of an anti-apoptotic protein of the Bcl-2 family.

48. The method of claim 47, wherein said anti-netrin-1 antibody and said antigen-binding fragment thereof exerts a tumor-specific Mcl-1 inhibition or degradation in said patient.

49. The method of claim 47, wherein said anti-netrin-1 antibody and said antigen-binding fragment thereof inhibits netrin-1 and UNC5B binding, and disengaged UNC5B makes that the CRL3-COMMD2 complex ubiquitinates Mcl-1, targeting it for proteasomal degradation.

50. The method of claim 47, wherein said cancer is a cancer having a percentage of Mcl-1 positive tumor cells which is equal or above grade 3, or equal to or above grade 4, or equal to grade 5, as measured by antibody staining using an antibody binding to Mcl-1, counting cancer cells in the cytoplasm and evaluating the percentage of staining positive cancer cells.

51. The method of claim 47, wherein said anti-netrin-1 antibody and said antigen-binding fragment thereof comprises
a variable domain VH comprising:
a H-CDR1 having a sequence set forth as SEQ ID NO: 28;
a H-CDR2 having a sequence set forth as SEQ ID NO: 29;
a H-CDR3 having a sequence set forth as SEQ ID NO: 30;
a variable domain VL comprising:
a L-CDR1 having a sequence set forth as SEQ ID NO: 31;
a L-CDR2 having a sequence set forth as SEQ ID NO: 32;
a L-CDR3 having a sequence set forth as SEQ ID NO: 9.

52. The method of claim 51, wherein the anti-netrin-1 antibody and antigen-binding fragment thereof is selected from the group consisting of anti-netrin-1 antibodies and antigen-binding fragments thereof comprising pairs of VH and VL amino acid sequences SEQ ID NO: 20 and 14, SEQ ID NO: 21 and 15, SEQ ID NO: 22 and 16, SEQ ID NO: 23 and 17, SEQ ID NO: 24 and 17, SEQ ID NO: 25 and 16, SEQ ID NO: 26 and 17, SEQ ID NO: 22 and 17, SEQ ID NO: 25 and 18, SEQ ID NO: 21 and 16, and SEQ ID NO: 27 and 19.

53. The method of claim 51, wherein the anti-netrin-1 antibody and antigen-binding fragment thereof comprises a VH of sequence of SEQ ID NO: 22 and a VL of sequence SEQ ID NO: 16.

54. The method of claim 51, wherein the anti-netrin-1 antibody further comprises a human IgG1 CH, and a human IgG1 CL Kappa.

55. The method of claim 47, wherein the inhibitor of an anti-apoptotic protein of the Bcl-2 family is a Bcl-2 inhibitor, in particular Venetoclax or Navitoclax.

56. A method of treatment of a patient suffering from a Mcl-1+ and netrin-1 receptor+, especially UNC5B+, tumor, comprising administering said patient with an efficient amount of an anti-netrin-1 monoclonal antibody or antigen-binding fragment thereof, and exerting a tumor-specific Mcl-1 inhibition or degradation.

57. The method of claim 56, wherein said anti-netrin-1 antibody and said antigen-binding fragment thereof inhibits netrin-1 and UNC5B binding, and disengaged UNC5B makes that the CRL3-COMMD2 complex ubiquitinates Mcl-1, targeting it for proteasomal degradation.

58. The method of claim 56, wherein said anti-netrin-1 antibody and said antigen-binding fragment thereof comprises
a variable domain VH comprising:
a H-CDR1 having a sequence set forth as SEQ ID NO: 28;
a H-CDR2 having a sequence set forth as SEQ ID NO: 29;
a H-CDR3 having a sequence set forth as SEQ ID NO: 30;
a variable domain VL comprising:
a L-CDR1 having a sequence set forth as SEQ ID NO: 31;
a L-CDR2 having a sequence set forth as SEQ ID NO: 32;
a L-CDR3 having a sequence set forth as SEQ ID NO: 9.

59. The method of claim 58, wherein the anti-netrin-1 antibody and antigen-binding fragment thereof is selected from the group consisting of anti-netrin-1 antibodies and antigen-binding fragments thereof comprising pairs of VH and VL amino acid sequences SEQ ID NO: 20 and 14, SEQ ID NO: 21 and 15, SEQ ID NO: 22 and 16, SEQ ID NO: 23 and 17, SEQ ID NO: 24 and 17, SEQ ID NO: 25 and 16, SEQ ID NO: 26 and 17, SEQ ID NO: 22 and 17, SEQ ID NO: 25 and 18, SEQ ID NO: 21 and 16, and SEQ ID NO: 27 and 19.

60. The method of claim 59, wherein the anti-netrin-1 antibody and antigen-binding fragment thereof comprises a VH of sequence of SEQ ID NO: 22 and a VL of sequence SEQ ID NO: 16.

61. The method of claim 56, wherein said patient is further treated with an inhibitor of an anti-apoptotic protein of the Bcl-2 family, preferably an inhibitor of the Bcl-2 protein.

62. The method of claim 56, wherein said tumor is a tumor having a percentage of Mcl-1 positive tumor cells which is equal or above grade 3, or equal to or above grade 4, or equal to grade 5, as measured by antibody staining using an antibody binding to Mcl-1, counting tumor cells.
